(19) United States
(12) Patent Application Publication
(10) Pub. No.: US 2021/0038228 A1
(43) Pub. Date: Feb. 11, 2021

(54) SYSTEMS AND METHODS FOR ATRIAL TRANSSEPTAL ACCESS AND CLOSURE

(71) Applicant: ReSept Medical, Inc., Los Altos, CA (US)

(72) Inventors: Chidambaram Rammohan, Los Altos, CA (US); Bernard Andreas, Los Altos, CA (US); John H. Morriss, Emerald Hills, CA (US)

(21) Appl. No.: 16/988,328

(22) Filed: Aug. 7, 2020

Related U.S. Application Data
(60) Provisional application No. 62/980,732, filed on Feb. 24, 2020.
(60) Provisional application No. 62/884,674, filed on Aug. 8, 2019.

(51) Int. Cl. A61B 17/12; A61B 17/04

(52) U.S. Cl. CPC A61B 17/12122; A61B 17/12031; A61B 2017/0409; A61B 2017/1205; A61B 17/0401

(57) ABSTRACT

A system for forming and closing an aperture in tissue for atrial transseptal access includes an elongate shaft with a tip at the distal end and a fastener platform coupled to the elongate shaft. The system also carries one or more fasteners with an anchor, and one or more penetrating shafts disposed alongside the elongate shaft. The fastener platform is deployable to an expanded configuration that engages and supports tissue surrounding the aperture. The one or more penetrating shafts may be extended through the tissue to engage a free end of the one or more fasteners so the fastener may be pulled through the tissue, or the penetrating shafts may pierce the tissue to deliver the fastener to a left side of the patient's heart for anchoring.

### CLAIM OF PRIORITY

The present application is a non-provisional of, and claims the benefit of U.S. Provisional Patent Application Nos. 62/884,674 (Attorney Docket No. 5635.001PRV) filed on Aug. 8, 2019; and 62/980,732 (Attorney Docket No. 62/980,732) filed on Feb. 24, 2020; the entire contents of which are incorporated herein by reference.

### BACKGROUND

Transseptal left atrial catheterization is a fundamental technique for cardiac electrophysiologic and structural cardiology. In transseptal procedures, a catheter is typically introduced percutaneously with the Seldinger technique or with a surgical cutdown into a vein such as the femoral vein and then advanced toward the right atrium of the patient's heart. An aperture is created in the atrial septum so that the catheter may be advanced from the right atrium to the left atrium where a therapeutic or diagnostic procedure is performed. Examples of procedures performed transseptally include, for example cardiac electrophysiologic mapping, ablation procedures to treat atrial fibrillation, left atrial appendage occlusion, transcatheter paravalvular leak closure, transcatheter mitral valve repair and replacement, as well as others.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a transseptal procedure and the basic anatomy of a human heart.

FIG. 2 illustrates an example of a transseptal access and closure system.

FIGS. 3A-3D show the fastener from FIG. 2.

FIGS. 4A-4Z shows an example of a transseptal access and closure system used on an atrial septum.

FIGS. 4Z1-4Z2 show an example of securing fasteners delivered by a transseptal access and closure.

FIGS. 5A-5E show another example of a transseptal access and closure system.

FIG. 6A shows a perspective view of another example of a transseptal access and closure system.

FIG. 6B shows a side view of the system in FIG. 6A.

FIGS. 7A-7Z4 show an example of a transseptal access and closure system used on an atrial septum.

FIG. 8A-8C show another example of a transseptal access and closure system used on an atrial septum.

### DETAILED DESCRIPTION

Transseptal left atrial catheterization is a fundamental technique for cardiac electrophysiologic and structural cardiology. In transseptal procedures, a catheter is typically introduced percutaneously with the Seldinger technique or with a surgical cutdown into a vein such as the femoral vein and then advanced toward the right atrium of the patient's heart. An aperture is created in the atrial septum so that the catheter may be advanced from the right atrium to the left atrium where a therapeutic or diagnostic procedure is performed. Examples of procedures performed transseptally include, for example cardiac electrophysiologic mapping, ablation procedures to treat atrial fibrillation, left atrial appendage occlusion, transcatheter paravalvular leak closure, transcatheter mitral valve repair and replacement, as well as others.

Residual intra-atrial communication is common after these procedures and may depend on the size of device used during the procedure. Patients often tolerate small intra-atrial residual flow in the short term when the flow is from the left atrium which has oxygenated blood to the right atrium which has de-oxygenated blood. However, when there is large flow from the left to the right atrium or when the flow is from the right atrium which has the de-oxygenated blood to the left atrium which has the oxygenated blood, there can be acute clinical decompensation where heart function deteriorates.

As the device sizes for transseptal procedures increase, the risk of residual atrial septal defects that are clinically significant increases. In some of these situations, the iatrogenic atrial septal defect can be closed with a transcatheter technique that delivers a closure device such as the Amplatzer® Septal Occluder or Gore® Cardioform. These closure devices address acute clinical decompensation by stopping the unwanted right to left and left to right blood flow across the atrial septum. However, there is significant risk from closure device thrombosis, embolization, erosion, etc. Furthermore, the closure device presence in the atrial septum can prohibit or significantly complicate re-access of the left atrium later on, especially in patients who have concomitant atrial arrhythmias, stroke risk, and complex valvular problems. Paravalvular leak closure after transcatheter mitral valve repair, valve-in-valve repeat transcatheter mitral valve repair are also common procedures that may require transseptal access. Thirty percent of atrial fibrillation ablations require a second procedure involving repeat transseptal access.

This inhibition of repeat transseptal access may be acceptable for those patients not expected to need additional transseptal procedures, such as end-stage salvage patients. But as new interventional technologies become more widely available and accepted for younger and lower risk patients, subsequent interventions will likely be needed. Therefore, it may be advantageous and desirable to repair transseptal openings using devices and methods that leave minimal hardware/implant materials behind in the atrial septum. It may also be desirable to provide devices and methods that minimize or prevent acute complications from right to left shunting or large left to right shunting as well as the long-term complications of moderate chronic left to right shunting without inhibiting future left atrial transseptal access. At least some of the objectives may be achieved by the examples of devices, systems and methods disclosed herein.

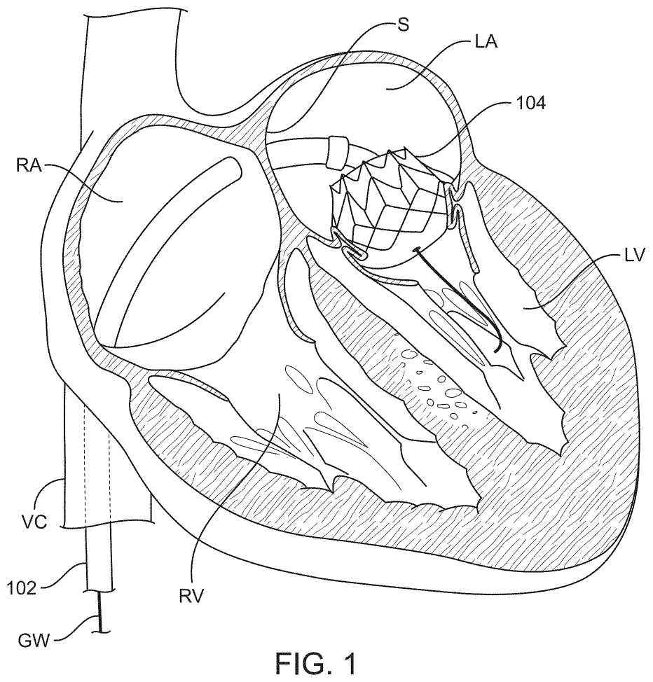

FIG. 1 shows the basic anatomy of human heart. The heart includes four chambers, the right atrium RA, left atrium LA, right ventricle RV, and left ventricle LV. Many interventional or diagnostic catherization procedures are conducted on the left side of the patient's heart. For example, FIG. 1 shows a guidewire GW in the patient's vena cava VC extending into the right atrium and passing across the atrial septum S into the left atrium, LA. The guidewire extends across the mitral valve into the left ventricle. A diagnostic or therapeutic catheter may then be inserted over the guidewire GW and advanced into the left side of the heart where a therapeutic or diagnostic procedure is performed. The guidewire may be introduced in a femoral vein in the groin or another vein via percutaneous introduction with the Seldinger technique, a surgical cutdown, or other techniques known in the art. In FIG. 1, a prosthetic mitral valve 104 is delivered by catheter 102 and the prosthetic valve is deployed into the native mitral valve. This route is referred to as a transseptal procedure and is often easier than other delivery routes such as transapically through the rib cage and apex of the heart, or a transaortic procedure where the catheter is delivered retrogradely through the aorta into the left heart. Because the atrial septum S is punctured, some of the challenges described above may be experienced during or after the procedure is performed. It therefore may be desirable to provide a device that can facilitate puncture and closure of the atrial septal puncture after the device is removed and the procedure is completed.

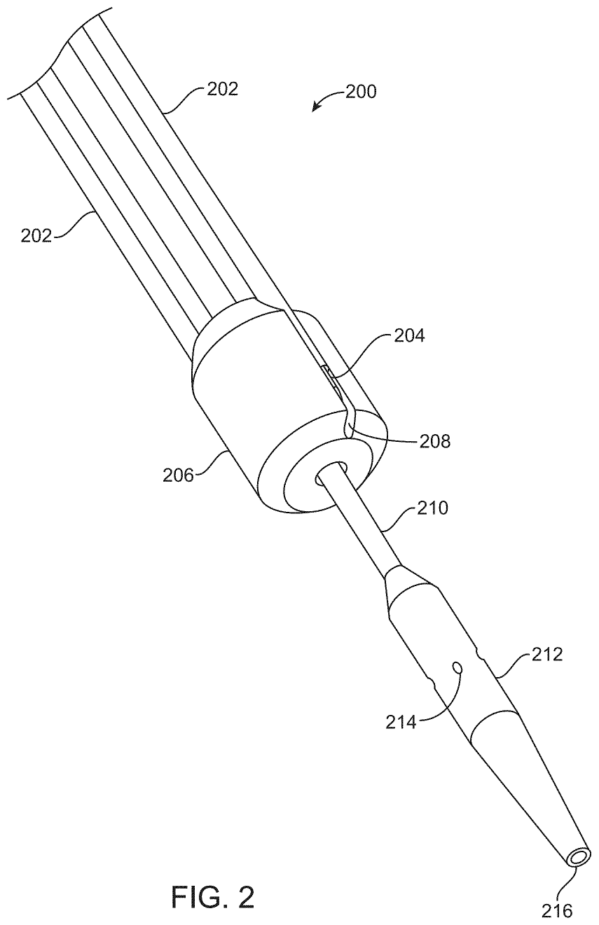

FIG. 2 illustrates an example of a transseptal access and closure system 200. The system 200 includes an elongate shaft 210 that has a proximal and distal end. A lumen extends the length of the elongate shaft 210. An atraumatic tapered tip 212 is coupled to the distal end of the elongate shaft 210. An aperture 216 in the distal end of tip 212 is fluidly coupled with the lumen in the elongate shaft 210 and is sized to slidably receive a guidewire. The tapered tip 212 also includes one or more aperture 214 which allow a platform to be extended radially outward as will be discussed below. A hub 206 is also coupled to a distal portion of the elongate shaft 210. The hub 206 includes open channels 208 that are sized and shaped to house a fastener delivery mechanism 204 that may be delivered to tissue surrounding an atrial septal puncture and used to help close the atrial puncture after the diagnostic or therapeutic procedure has been completed. In the example of FIG. 2, two fastener delivery mechanisms are shown but this is not intended to be limiting and three, four, or more fasteners may be delivered. The fasteners are carried in sheaths 202. The elongate shaft 210 and sheaths 202 extend proximally back to a handle that will be discussed later. Optionally, an outer sheath may cover the sheaths 202, hub 206, and tip 212 to provide protection.

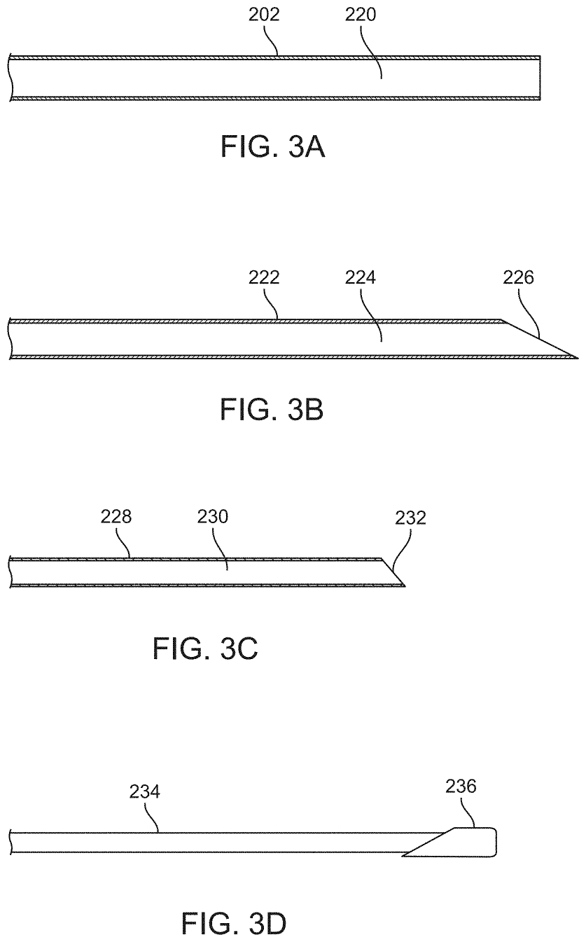

FIGS. 3A-3D illustrate the fastener delivery mechanism 204 used in FIG. 2. The fastener includes a sheath 202 seen in FIG. 3A, a needle 222 in FIG. 3B, a pusher 228 in FIG. 3C, and the fastener 234 shown in FIG. 3D.

FIG. 3A shows sheath 202 which is an elongate tube with a lumen 220 extending back to a handle as will be described later. The sheath 202 houses the needle 222, pusher 228, and fastener 234 concentrically in the lumen 220.

FIG. 3B shows the needle 222 which is slidably disposed in the lumen 220 of sheath 202. The needle 222 includes a tissue piercing tip 226 and a lumen 224. The needle also extends proximally to the handle which will be discussed later.

FIG. 3C shows the pusher 228 which is slidably disposed in the lumen 224 of the needle 222. Pusher 228 has a lumen 230 and also a beveled distal tip 232 that facilitates actuation of the fastener as will be discussed later. The pusher extends proximally to a handle that will be described in more detail later.

FIG. 3D shows the fastener 234 which in this example is a filament such as a wire or a suture and includes an actuatable anchor 236 coupled to the distal end of the fastener 234. The fastener is shown in a collapsed configuration but may be actuated into an expanded configuration as will be shown later. The fastener 234 is sli dably disposed in lumen 230 of pusher 228 and extends proximally to the handle that will be discussed later.

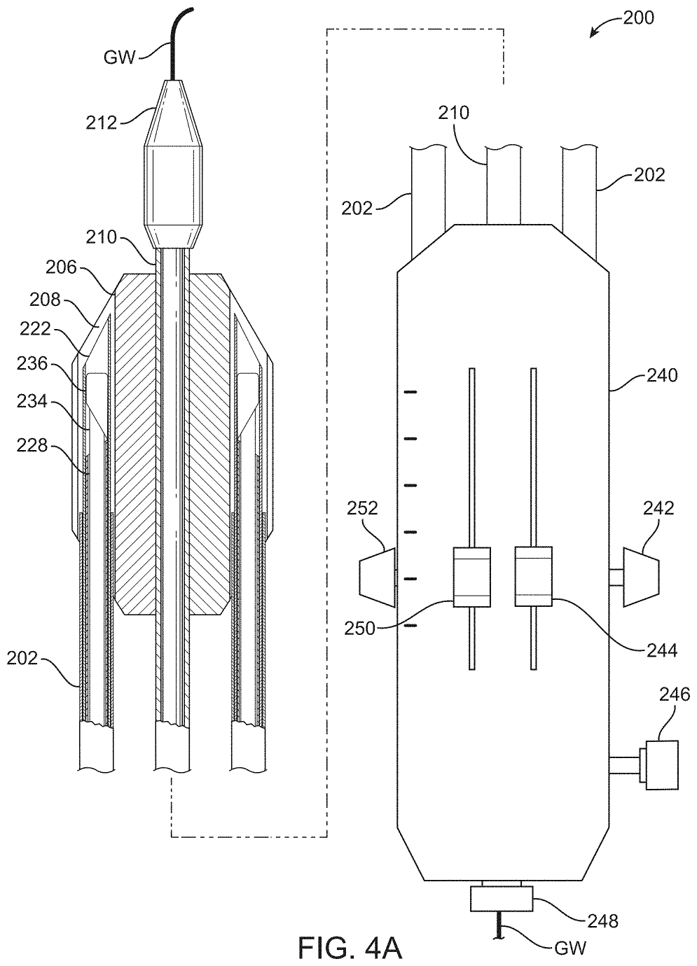

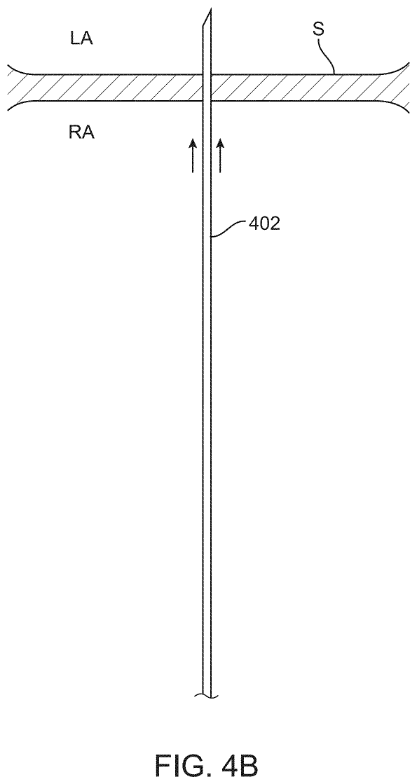

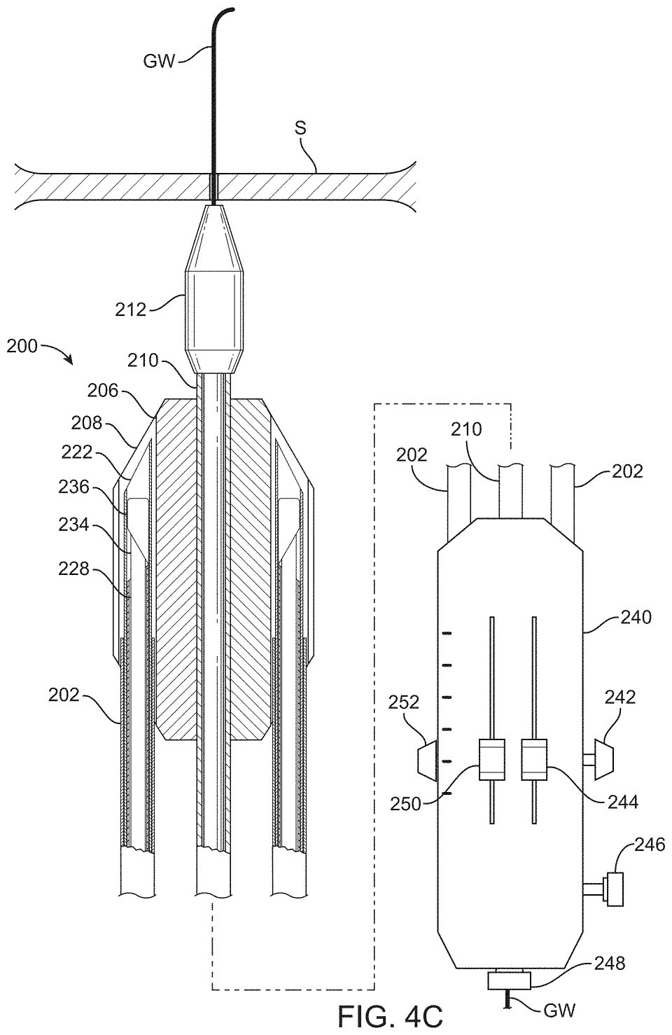

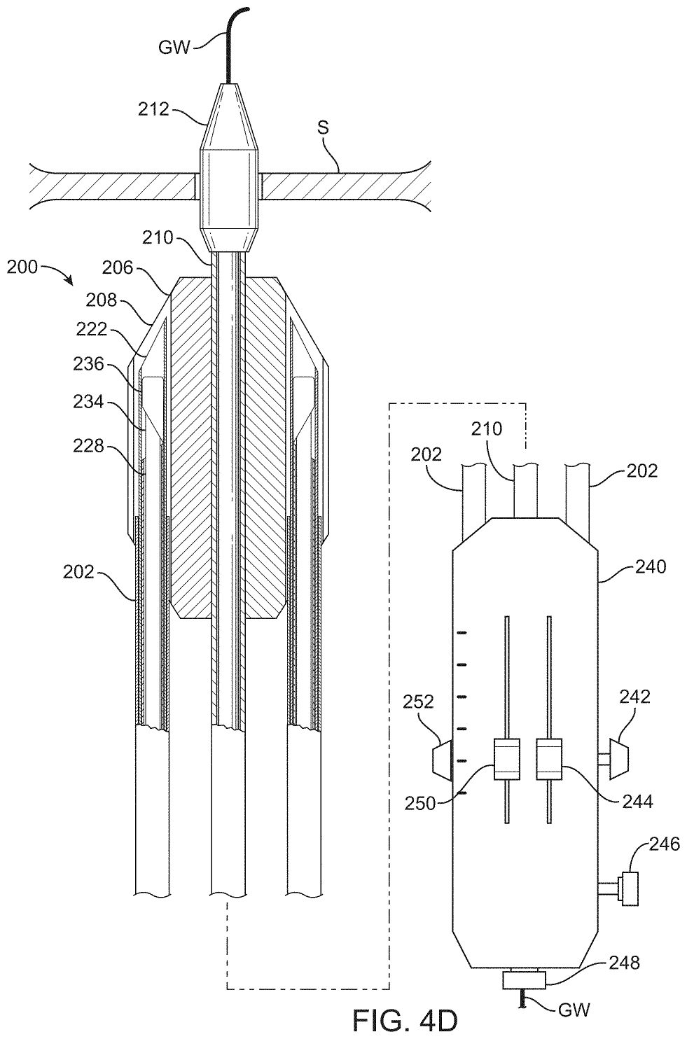

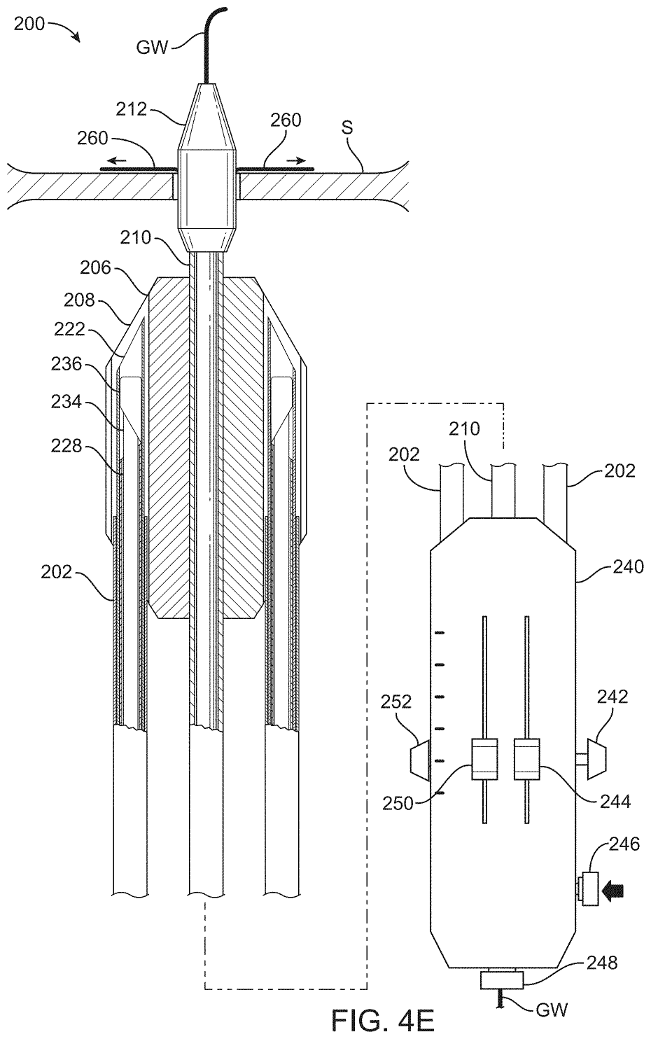

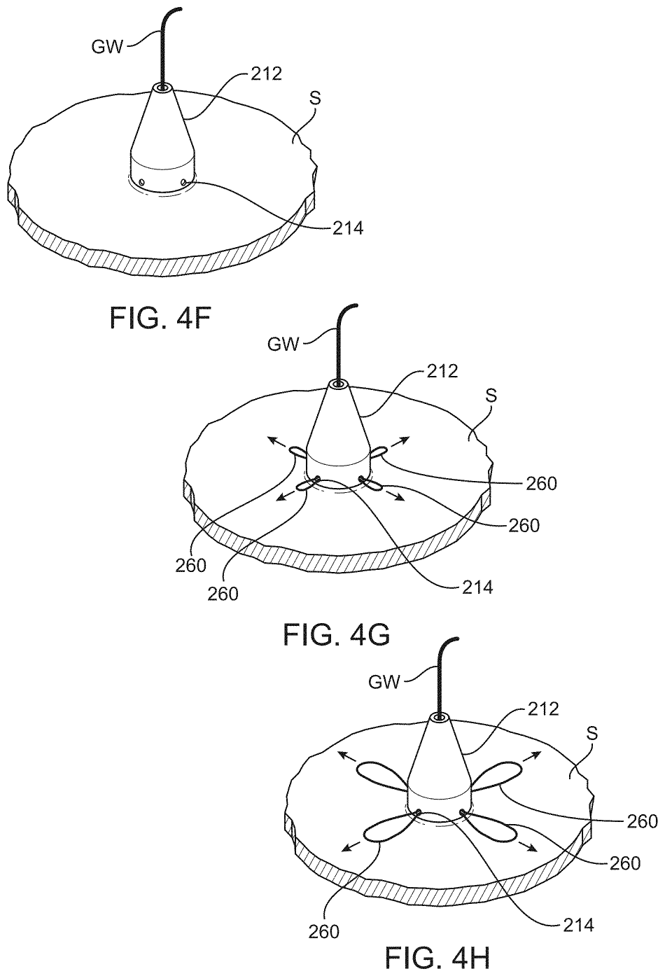

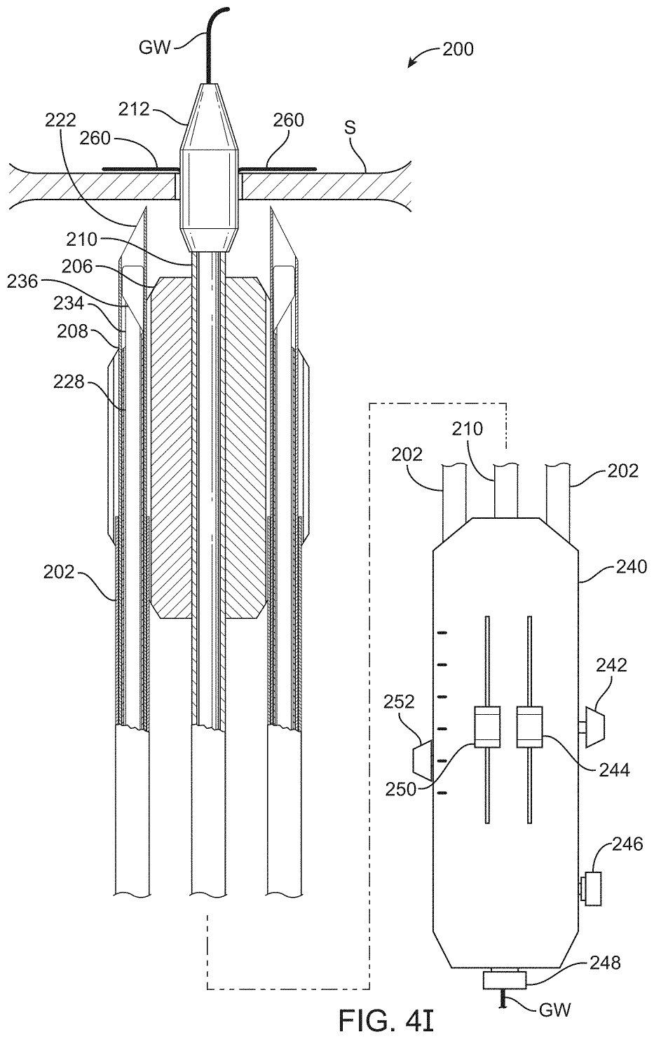

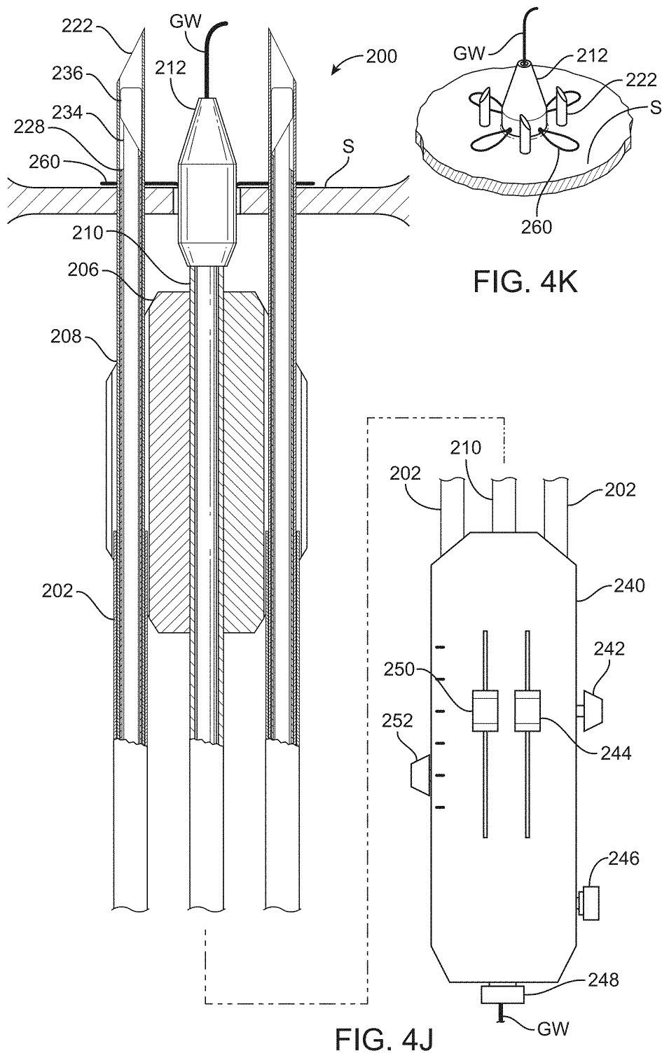

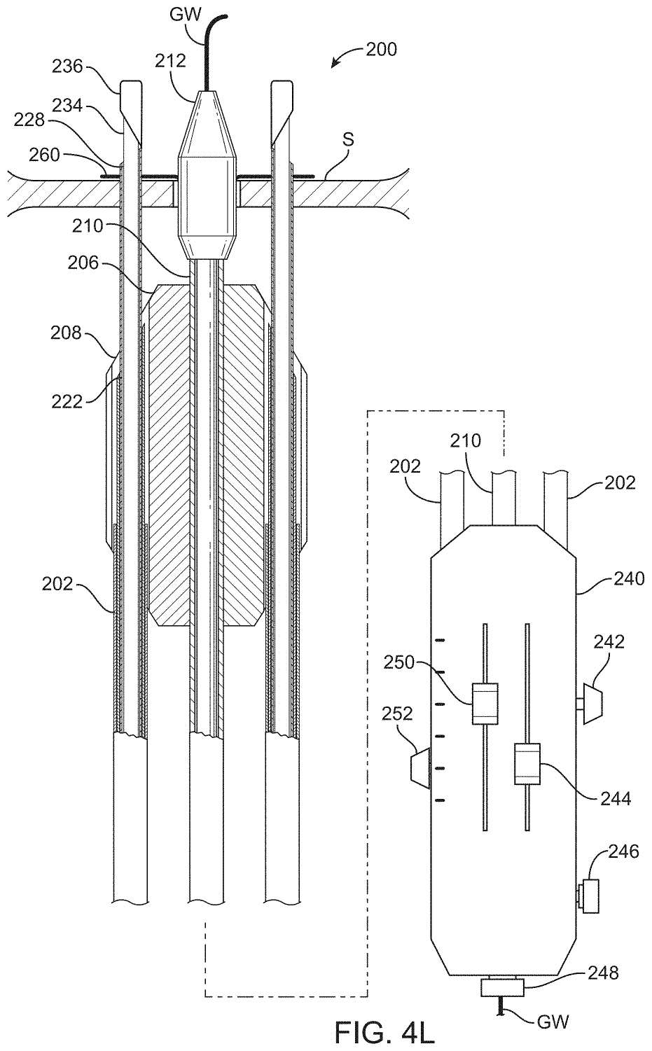

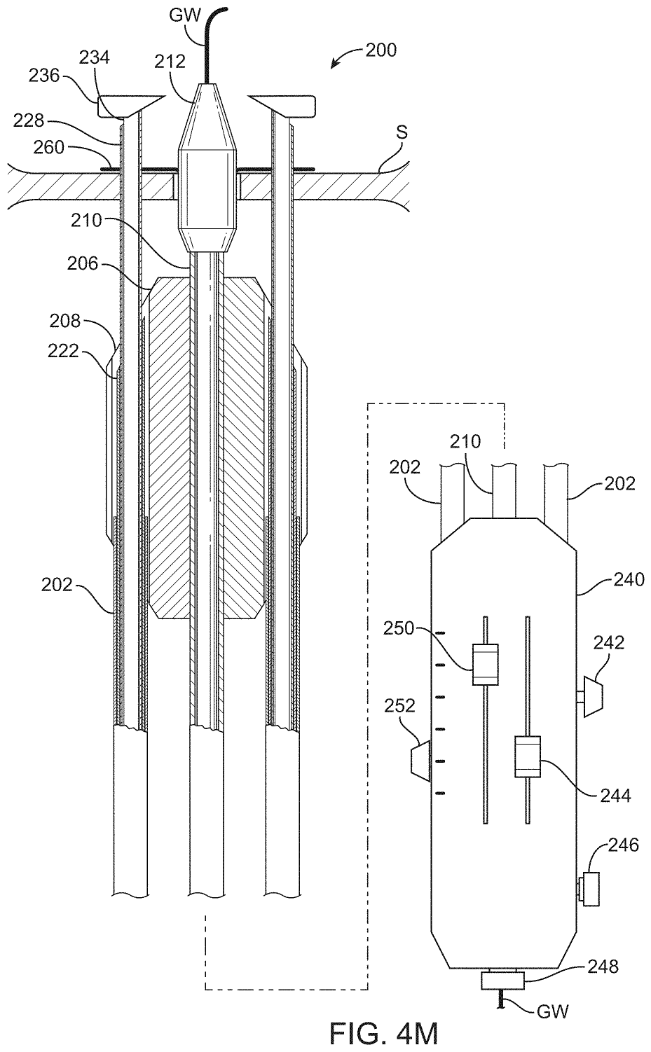

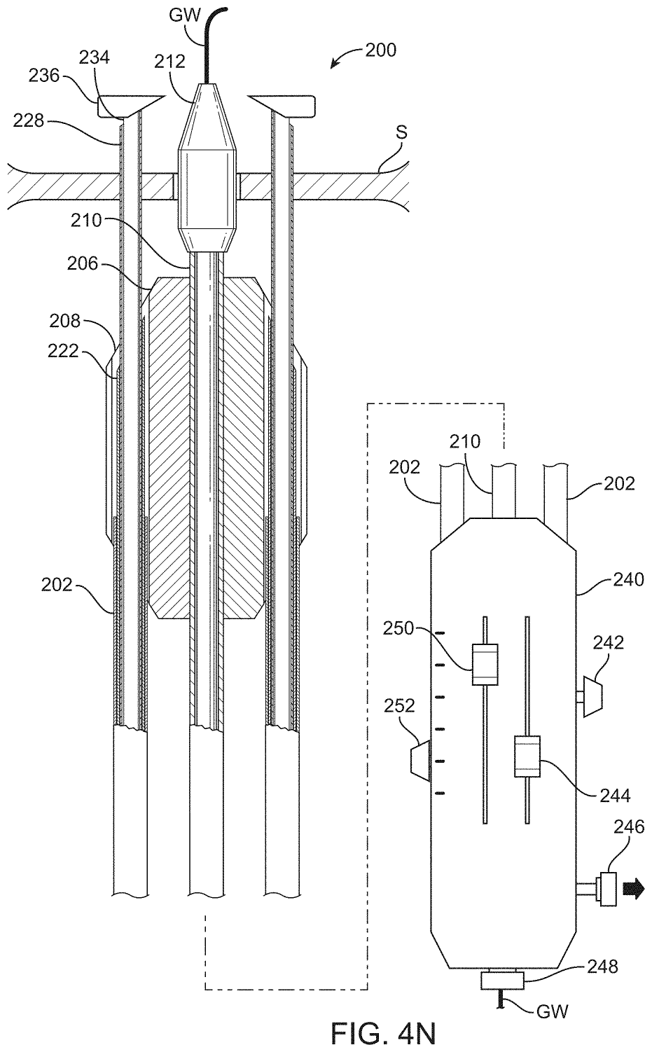

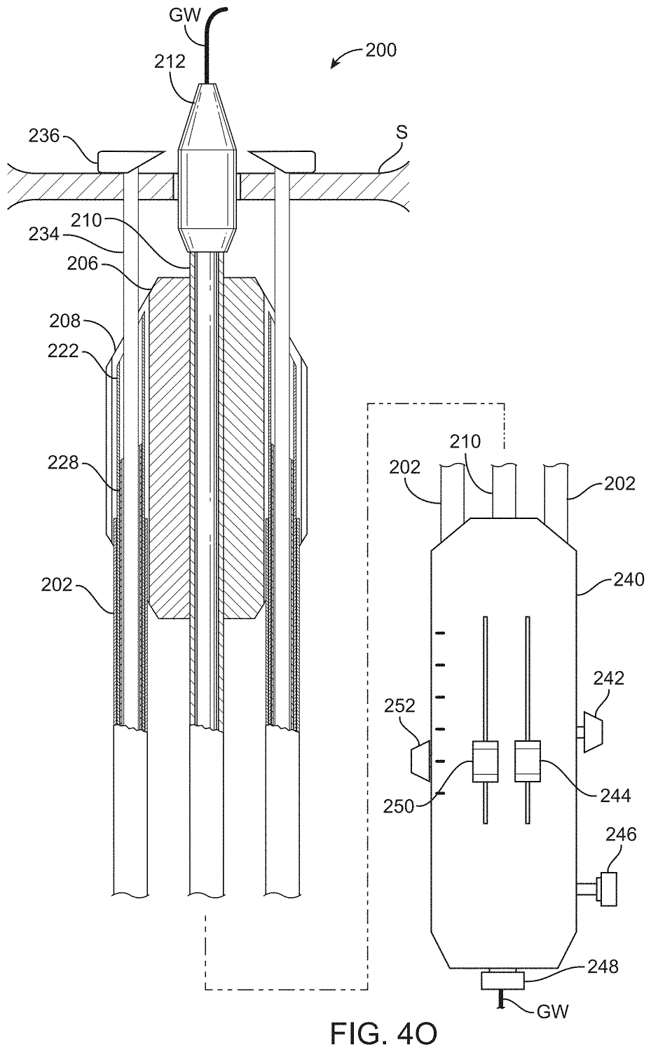

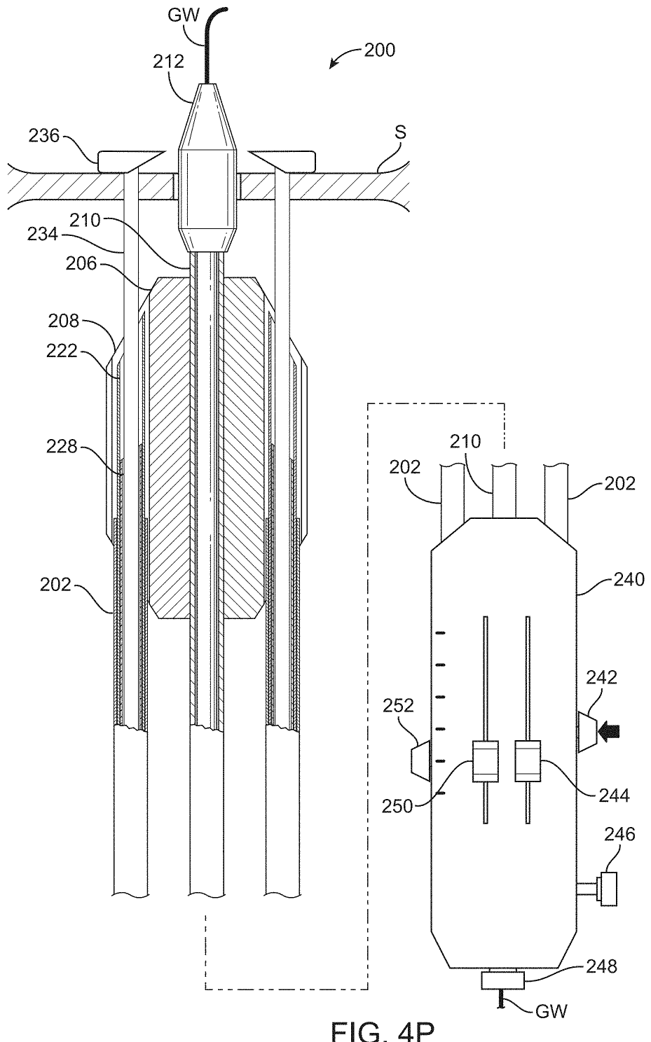

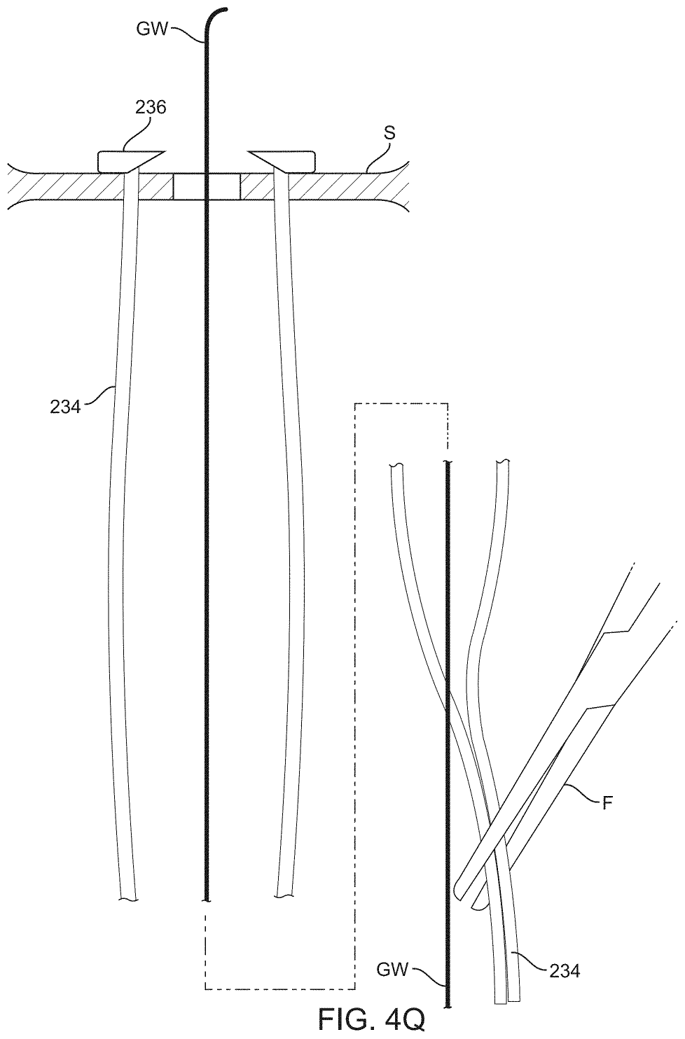

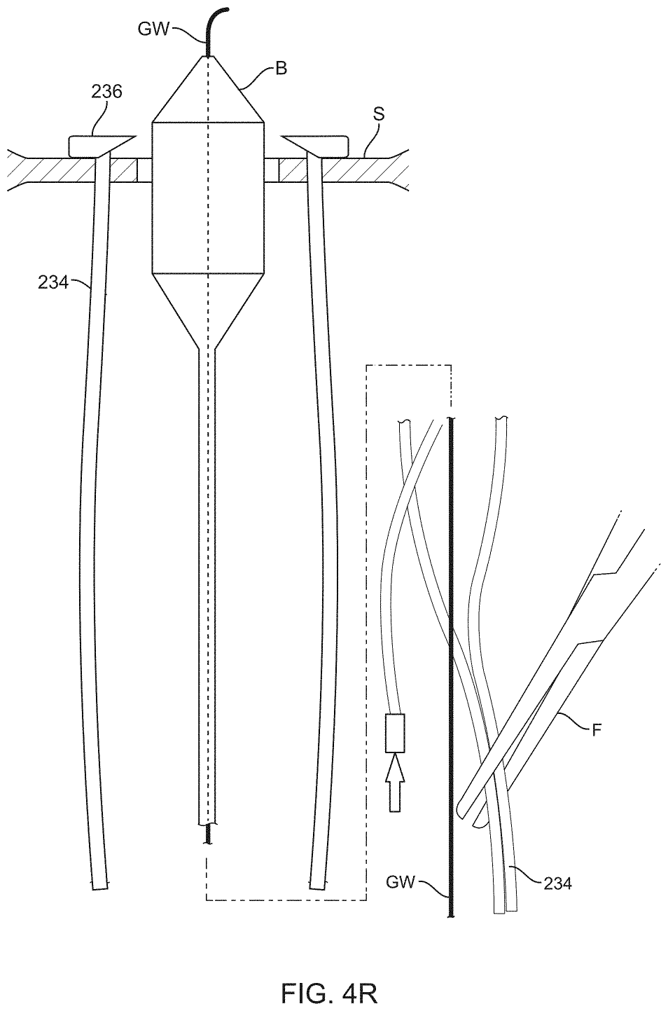

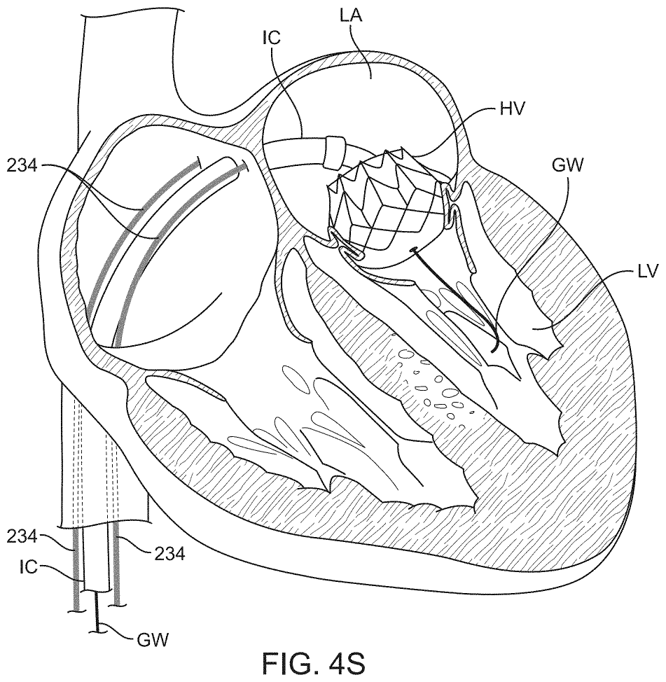

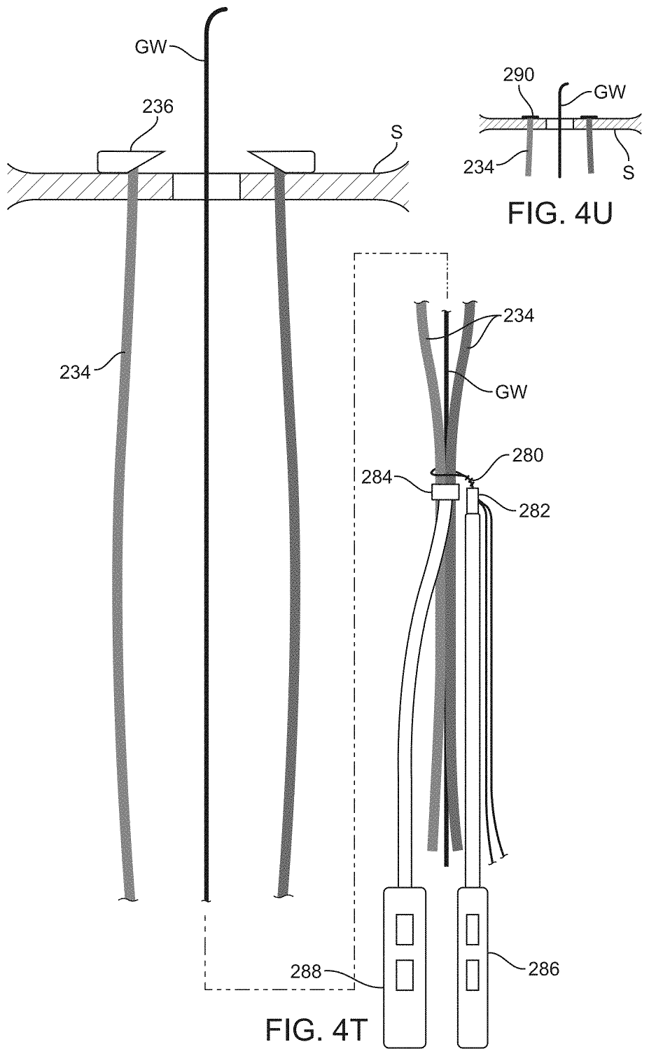

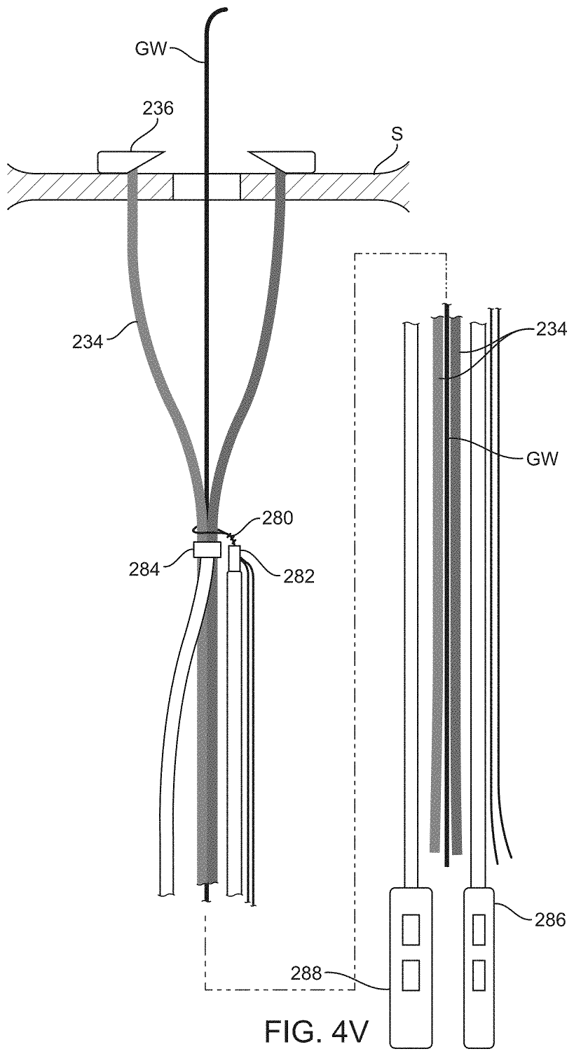

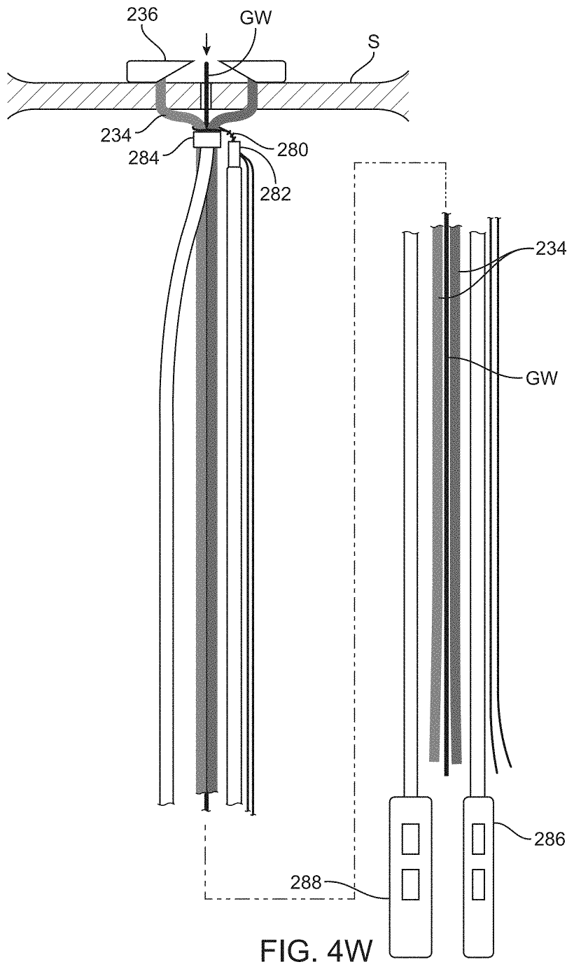

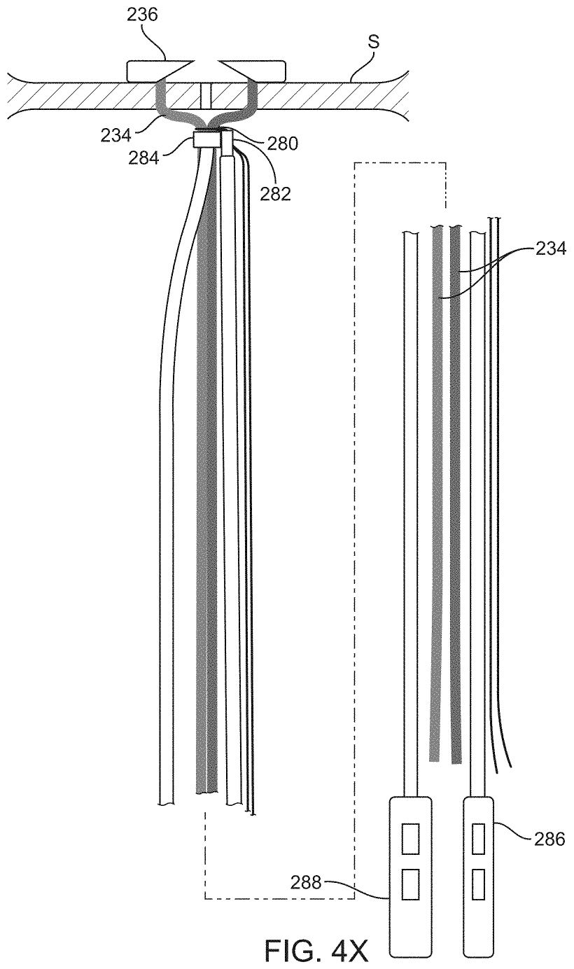

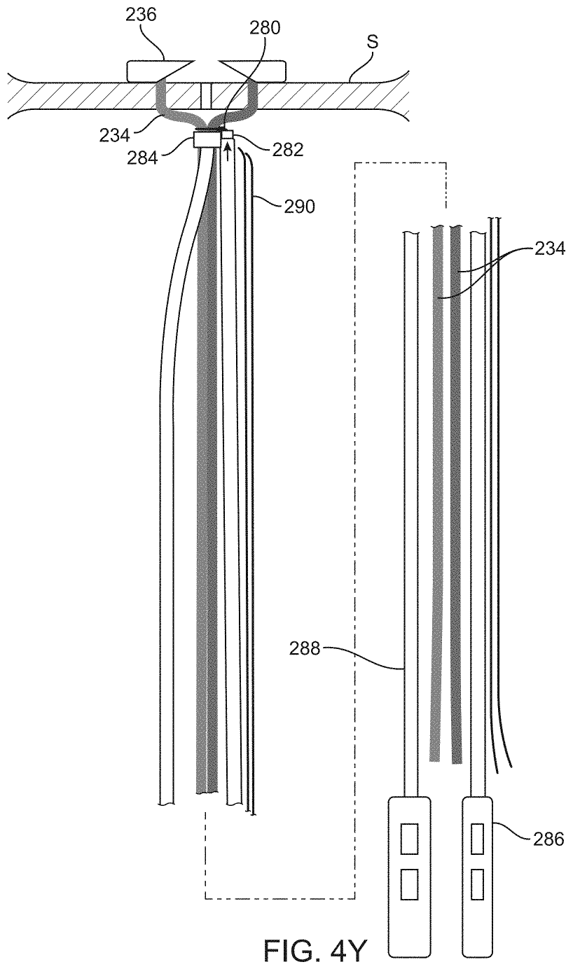

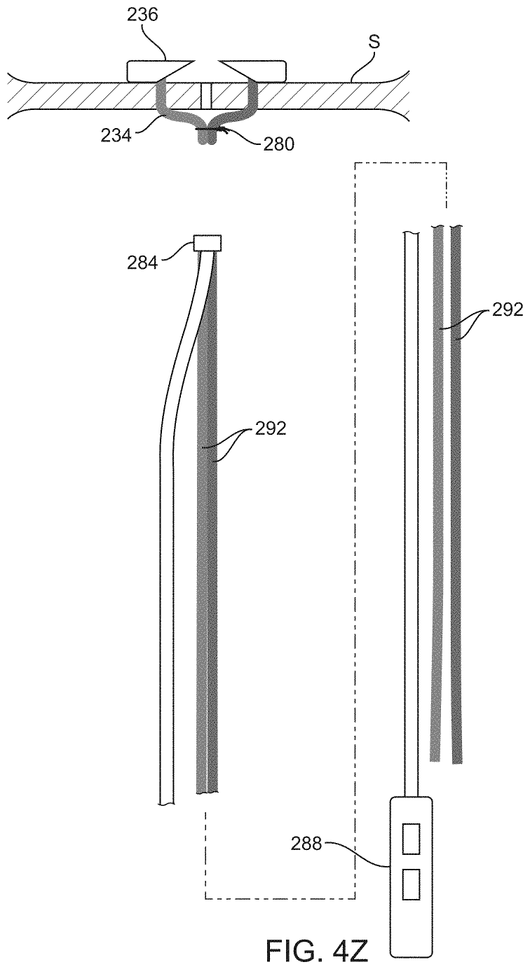

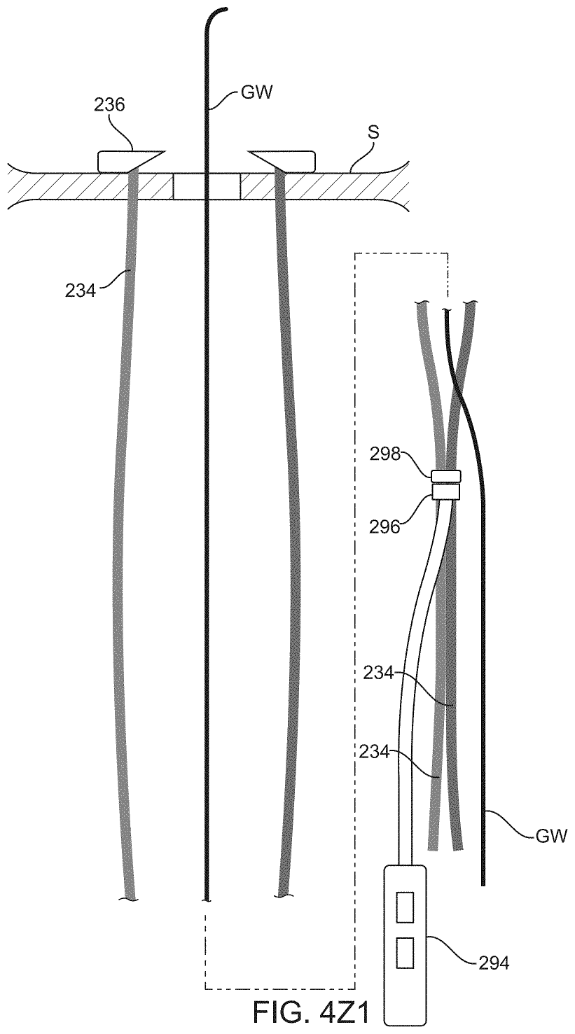

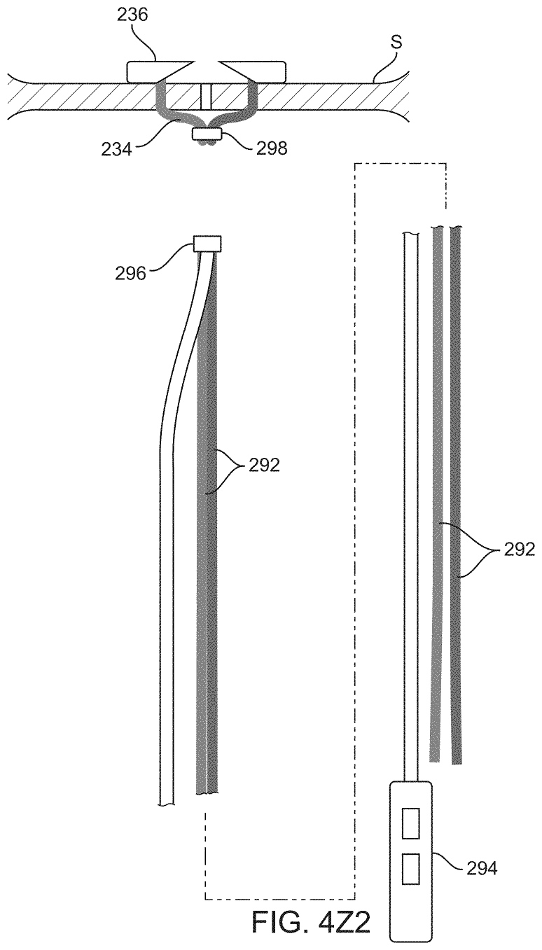

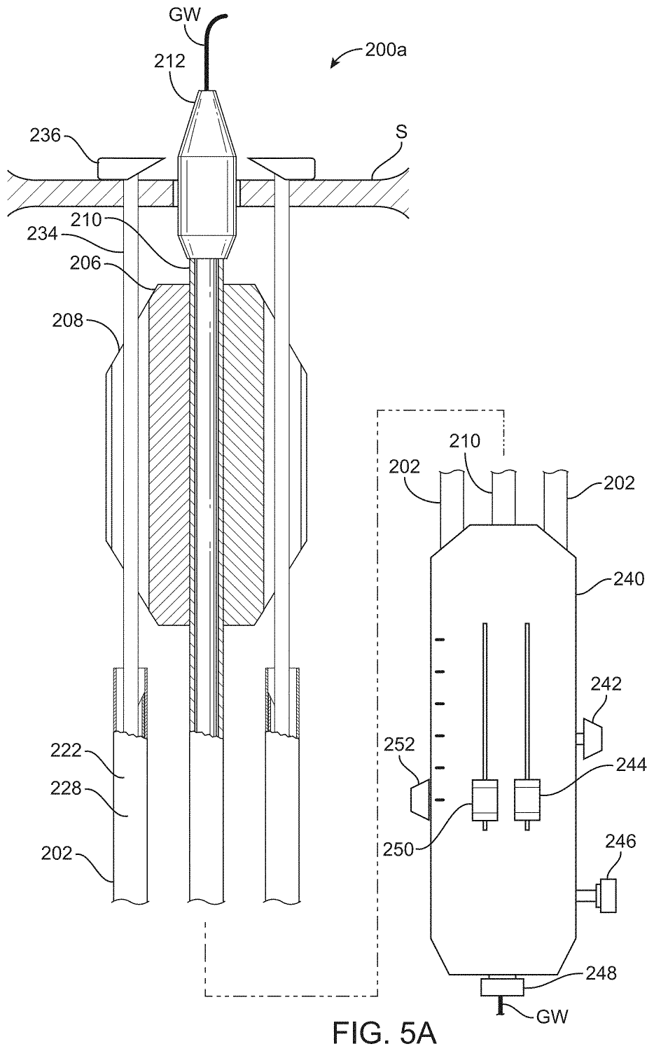

FIGS. 4A-4Z show an example of the transseptal access and closure system 200 shown in FIG. 2 used to close an atrial septal puncture.

In FIG. 4A, the system 200 is delivered over a guidewire GW. A central elongate shaft 210 includes an atraumatic tapered distal tip 212 coupled to the distal end of the elongate shaft 210. A hub 206 is also coupled the elongate shaft 210 near its distal end. The hub includes open channels 208 that house the fastener delivery mechanism 204 which includes a sheath 202 concentrically disposed over a needle 222 which is concentrically disposed over pusher 228 which is concentrically disposed over fastener 234 with expandable anchor 236. There are four sheaths 202, four needles 222, four pushers 228 and four fasteners 234 each with an expandable anchor 236 but FIG. 4A only shows two in the view presented.

The sheaths, here there are four sheaths 202 and the elongate shaft 210 extend proximally and are operatively coupled with a handle 240 that includes several actuators for controlling movement of the various components of the system. For example, optional slider 252 controls movement of sheaths 202, slider 250 controls movement of pusher 228, slider 244 control movement of needle 222. Other actuators include a fastener release button 242 that releases the fasteners 234 from the handle 240, and button 246 may be used to deploy a fastener platform as will be discussed in greater detail below. Indicia such as a scale, or other markings on the handle allow an operator to relate actuator position with the movement of the various components.

The system of FIG. 4A may be used to facilitate access across the septal wall and also may be used to facilitate closure of the septal puncture after the procedure is complete.

FIG. 4B shows the initial step in a transseptal access and closure procedure that may be performed using the system in FIG. 4A. Here, a tissue piercing needle is advanced from the right atrium RA to the left atrium LA by piercing through the attial septum S. The needle 402 maybe a Brockenbrough needle or other needle used by cardiologists. The needle may be advanced over a guidewire and once the septal wall is pierced, the guidewire may be advanced across the septal wall and the needle retracted proximally. Here the piercing needle is a separate device from the transseptal access and closure device, while in other examples below the piercing needle is integral with the system.

FIG. 4C shows that after the atrial septum has been pierced and a guide wire advanced across the septum, the transseptal access and closure system 200 may be loaded over the guidewire GW and advanced distally. The distal tip 212 is advanced until it is adjacent the hole in the septum S on the right side of the heart, with the rest of the working end of system 200 still in the right heart.

FIG. 4D shows the entire system 200 advanced distally so that the tapered distal tip 212 is advanced through the hole in the septum S. Distal advancement is performed by the operator pushing the handle 240 distally so that the entire system moves distally. The tapered tip is advanced distally enough so that the apertures 214 (best seen in FIG. 2) are on the left side of the heart in the left atrium.

FIG. 4E shows deployment of platform 260 from the tapered distal tip 212. Here, actuation of button 246 as indicated by the arrow delivers a force to the platform 260 causing it to radially expand outward and away from the tapered tip 212. Here, wire filaments such as nitinol wires are stored in the tapered tip 212 and the wire filaments are coupled to a rod that may be slidably disposed in the lumen of elongate shaft 210. The rod (not shown) is then coupled with button 246. Thus actuation of button 246 will either apply a compressive force to the rod which deploys the wire filaments, or actuation of button 246 in the opposite direction will apply a tensile force to the rod causing the wire filaments to contract back into the tapered tip where they are stored. Here, the filaments are wings or petal-like looped shapes. Any number of petals may be employed, here four petals form the platform with two petals visible while the other two are not seen in FIG. 4E. The platform is then disposed against the septal wall on the left side of the heart and provides a backstop to support the septal wall when additional piercings through the septal wall are formed by the system 200 as will be described below. In some situations, the entire system may need to be retracted slightly in a proximal direction to ensure that the platform 260 is apposed with the septal wall.

FIG. 4F shows the tapered tip 212 being advanced distally over the guidewire GW so that the apertures 214 are disposed on the left side of the heart in the left atrium.

FIG. 4G shows partial deployment of the platform 260 from the apertures 214 in the tapered tip 212. Here, the platform is formed from four looped petals that are stored in a collapsed configuration in the tapered tip 212. Actuation of button 246 pushes the petals out of the tapered tip 212 into an expanded configuration where the loops expand radially outward and provide a support surface.

FIG. 4H shows full expansion of the platform 260 with the petals extended fully to form the support surface. The petals may also be referred to as wings herein. The loops of the petals abut with the surface of left side of the atrium septum.

In FIG. 4I, slider 244 is operatively coupled to needle 222, and slider 250 is operatively coupled to pusher 228. Both sliders 250, 244 are advanced distally to move the assembly of the needle 222, pusher 228, and the fastener 234 (here a suture) with actuatable anchor 236, all move distally together toward the right side of the atrial septal wall S. Advancement of the pusher advances the fastener 234 because the pusher pushes against the actuatable anchor 236 which is coupled to the fastener 234. The sheath 202 remains in position.

FIG. 4J shows further actuation of sliders 250, 244 extend the needle 222, pusher 228, and the fastener 234 with actuatable anchor 236 distally until the needle pierces through the septal wall S and the actuatable anchor 236 is disposed on the left side of the heart in the left atrium while still disposed in the needle.

FIG. 4K shows a view of the needles 222 penetrating through the septal wall S, as viewed from the left atrium of the heart. Here, there are four needles although only three are visible. Platform 260 provides support for the tissue so that the needles 222 can pierce through the septal wall S tissue without the tissue bowing away from the needles. The needles pass in the gaps between adjacent petals or wings of the platform 260.

In FIG. 4L, after the actuatable anchor 236 has been properly positioned on the left side of the heart, slider 244 may be retracted proximally to retract needle 222 away from the fastener 234 and actuatable anchor 236. The needle 222 may be retraced back to the right side of the heart in the right atrium and the needle may be retracted back into sheath 202 which is disposed in open channel 208 of hub 206 so the sharp piercing tip is unexposed. This leaves a distal portion of the fastener 234 disposed on the left side of the heart with the actuatable anchor 236 also disposed on the left side of the heart. The distal end of pusher 228 is also disposed on the left side of the heart.

FIG. 4M shows distal advancement of slider 250 to move pusher 228 distally so that the distal tip of pusher 228 engages the actuatable anchor 236. Actuatable anchor 236 is pivotably coupled with fastener 234 so that as pusher 228 is advanced distally, the pusher 228 forces the anchor 236 to pivot and deploy so that its longitudinal axis is transverse or orthogonal to the longitudinal axis of the fastener 234. The length of the fastener 236 is longer than the diameter of the hole pierced in the septal wall S, therefore the pivotable anchor will not be able to pass back through the septal wall when tension is applied to the fastener 234, here a suture. Thus, fastener 234 now has one end anchored to the septal wall S on the left side of the heart in the left atrium. And the other anchors are also similarly anchored.

FIG. 4N shows after the actuatable anchors have been properly deployed on the left side of the heart, the platform 260 may be retracted so that it returns to a collapsed configuration and is housed in the tapered tip 212. Actuation of actuator button 246 in the opposite direction to the initial actuation retracts the petals back into the tapered tip 212.

FIG. 4O shows proximal retraction of slider 250 which proximally retracts the pusher 228 back into sheath 202 which is disposed in the open channel 208 of hub 206. The fastener, here a suture 234 is now anchored with anchor 236 to the left side of the heart. The suture passes through the septal wall S and the fastener extends proximally back to the handle 240. The length of the fastener 234 between the open channel 208 in hub 206 and the right side of the septal wall S is unconstrained and floats freely in the heart. The rest of the fastener is disposed in sheath 202 where it is protected. Also, the entire system 200 may be pulled back proximally so that the anchors 236 are apposed with tissue surrounding the septal piercing.

FIG. 4P shows actuation of actuator 242, here a button, after the fasteners 234 and anchors 236 have been properly placed and the needles 222 and pushers 228 have been retracted. The free ends of fasteners 234 are coupled to the handle and actuation of button 242 releases the free ends of fasteners 234 from the handle 240. Actuation of button 242 may open a clamp that holds the free ends, or may actuate a cutting element that severs the free ends, or other release mechanisms may be used.

FIG. 4Q shows that once the free ends of the fasteners 234 have been released from the handle 240, the handle 240 may be retracted proximally drawing the entire system proximally until it is removed from the body, except for the fasteners 234 which are anchored with anchors 236 to the septal wall. The free ends of the fasteners are disposed outside the body and the free ends may be clamped together with a clip, forceps F, or other clamps.

After the fasteners have been secured to the atrial septum, a separate balloon catheter B may be advanced over the guidewire and disposed in the septal puncture as shown in FIG. 4R. Inflation of the balloon then further dilates the septal puncture to allow a diagnostic or interventional device to be delivered transseptally into the left atrium. The balloon may be integral with the transseptal access and closure device or the balloon may be a separate and discrete balloon catheter that is delivered separately from the access and closure system. Additional disclosure related to the balloon dilation catheter is disclosed later in this specification.

FIG. 4S shows that once the septal puncture has been enlarged by balloon dilation, an interventional catheter IC or diagnostic device maybe advanced over the guidewire GW transseptally into the left atrium LA. In this example, a prosthetic mitral heart valve HV is delivered with the interventional catheter IC and deployed in the native mitral valve. Other interventional devices may also be used including but not limited to valvuloplasty devices, angioplasty devices, ablation devices, other prosthetic valves, annuloplasty rings, etc. The fasteners 234 remain anchored to the septal wall, and they run alongside the interventional catheter IC and exit the body where their free ends are clamped together.

After the diagnostic or interventional procedure is complete and the diagnostic or interventional catheter has been removed, the puncture may be closed. FIG. 4T shows the use of a knot instrument 286 and a fastener cutter instrument 288 to help secure the fasteners and close the septal puncture. Here, a knot 280 is attached to a tip of the knot instrument 286 which also includes a knot cutting element 282. The fastener cutter instrument 288 also includes a cutting element 284 near its distal end. The knot 280 is disposed over the fasteners 234 and is pushed distally by advancing the knot instrument 286 distally as well as optionally using the fastener instrument 288 to help push the knot distally. Actuators on both the knot instrument 286 and the fastener cutter instrument 288 may be actuated to cut excess suture in the knot 280 as well as the excess fastener ends 234.

The example shown in FIGS. 4A-4T has focused on the use of fasteners which are single strands of suture or single filaments with one end anchored to the septal wall by an actuatable anchor. However, this is not intended to be limiting. Other anchors may be used instead of the actuatable anchor. For example, FIG. 4U shows an example where each single filament has a T-shaped end 290 which may be introduced similarly as described above with the major difference being that the T-shaped end is housed in the needle with the head of the T laying parallel with the longitudinal axis of the needle and then the pusher pushes the T head out of the needle where it returns to its unbiased position with the T head being substantially orthogonal to the elongate fastener, thereby anchoring the fastener to the atrial septal wall.

FIG. 4V shows further advancement of the knot 280 distally toward the atrial septum S by pushing the knot instrument 286 and cutting instrument 288 distally.

FIG. 4W the knot 280 is advanced further distally until it is adjacent the piercing in the septal wall S, thereby tensioning the fasteners 234 which will close the aperture pierced in the septal wall. The knot is advanced distally by pushing the knot instrument 286 and the cutting instrument 288 further distally so that the knot slides distally over the fasteners 234. The guidewire GW may be left in place until the operator is assured that the septal aperture is ready to be closed and then the guidewire GW is retracted proximally to remove it from the pierced hole in the septal wall S.

FIG. 4X shows the guidewire GW removed. The knot 280 is further cinched tightly by advancing the knot instrument 286 distally so that the hole pierced in the septum is closed or nearly closed. The cutting instrument 288 is also advanced distally so the cutting element is adjacent the septal wall S.

FIG. 4Y shows that after the fasteners have been tensioned to a desired amount closing or substantially closing the hole pierced in the septum 5, the knot instrument 286 may be actuated by actuating an actuator such as a button or slider on the handle to actuate a knot cutting element 282 to cut the excess suture 290 from the knot 280 and allowing the excess suture 290 to be removed from the patient. The knot instrument 286 may then be retracted proximally and removed from the patient. Similarly, the cutting instrument 288 may also be actuated by actuating an actuator such as a button or slider on the handle to actuate cutting element 284 to cut the excess fastener 234 from the knotted portion secured to the septal wall with knot 280 and anchors 236. The excess fastener material may then be removed from the patient.

FIG. 4Z shows knot instrument 286 and excess suture 290 removed from the patient. The cutting instrument 288 is retracted proximally away from the septal wall S along with the excess portions of fastener 292. This leaves fastener 234 secured to septal wall S with knot 280 and anchors 236, thereby closing or substantially closing the hole pierced in the septum S. The cutting instrument 288 and excess portions of fastener 292 are then removed from the patient.

FIG. 4Z1-4Z2 show the use of a clip or crimping element to secure the fasteners instead of, on in conjunction with the knot shown in FIGS. 4S-4Z above. In FIG. 4Z1, after the fasteners 234 have been anchored with anchors 236 to the septal wall S and the access and closure system has been removed, the guidewire may remain in position, positioned in the hole pierced through the septal wall S. A clip instrument 294 with a clip or crimping element 298 is advanced over the fasteners 234 toward the septal wall. The clip instrument also may include a clip actuator or crimping actuator 296. The clip is not advanced over the guidewire GW so that the guidewire is not fastened to the fasteners.

In FIG. 4Z2, after the clip or crimping element 298 has been advanced distally to apply a desired amount of tension to the fasteners 234 so that the puncture in the atrial septum S is closed or substantially closed, the guidewire GW is removed. Then the clip actuator or crimping actuator 296 is actuated by an actuator on the handle of the clip instrument 294, thereby clamping the fasteners 234 or crimping them into position. The clip actuator or crimping actuator 296 may also include a cutting element that can cut the excess strands of the fastener 292 so they may be removed from the patient. The clip instrument 294 and excess portions of the fastener 292 are then removed from the patient and the puncture site is held in a closed of substantially closed position.

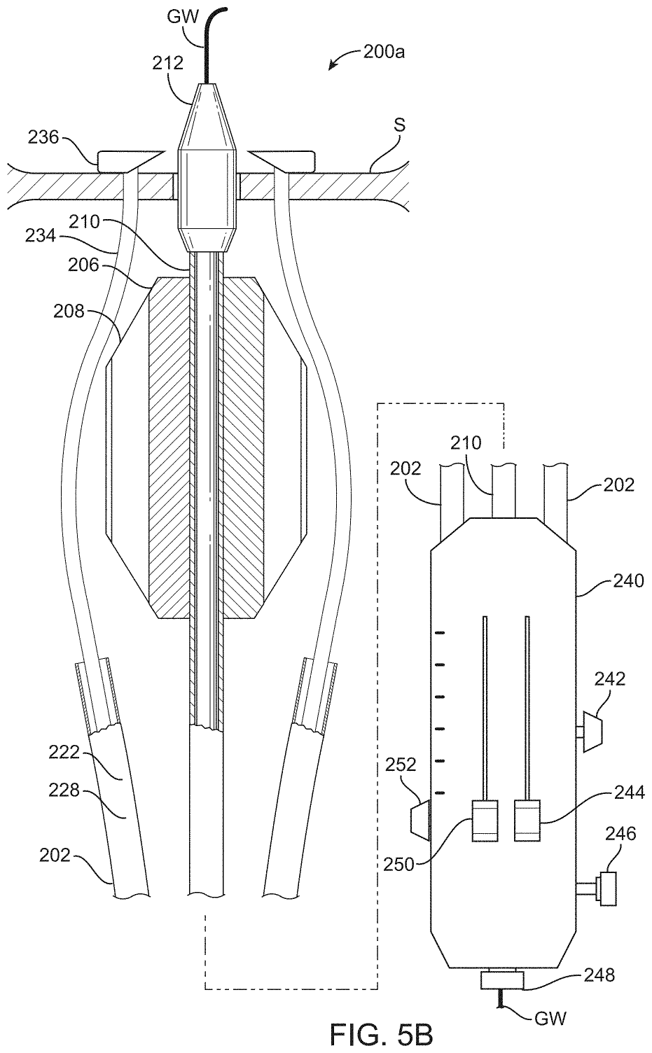

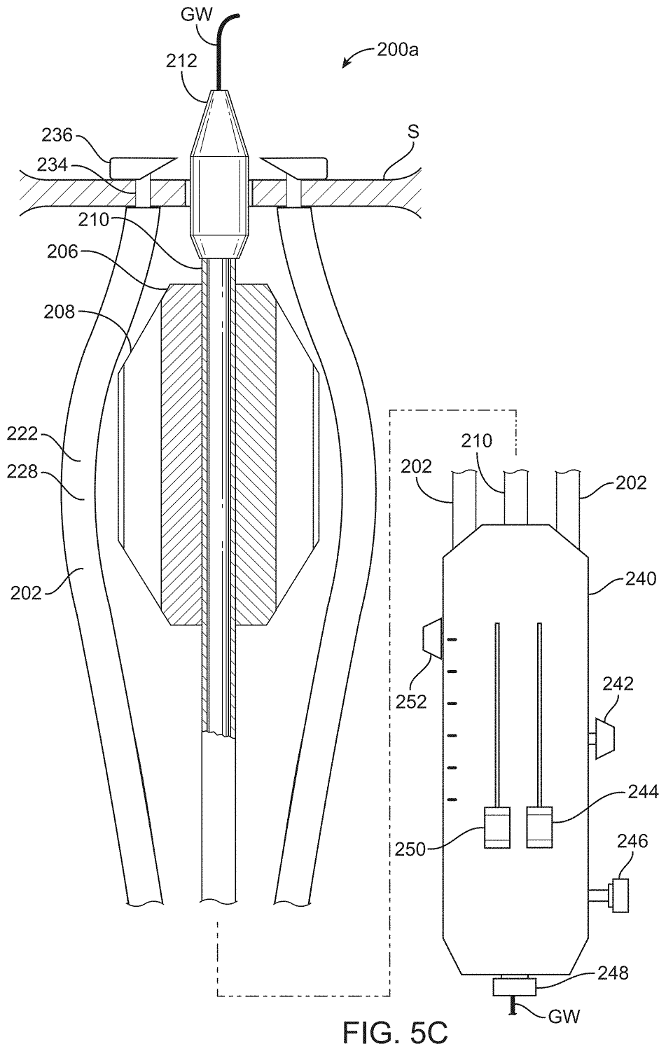

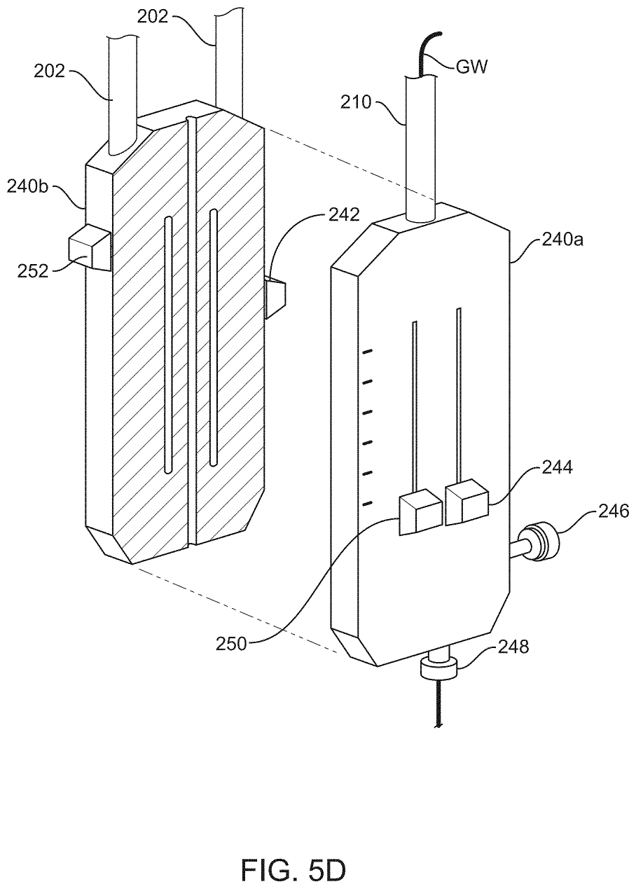

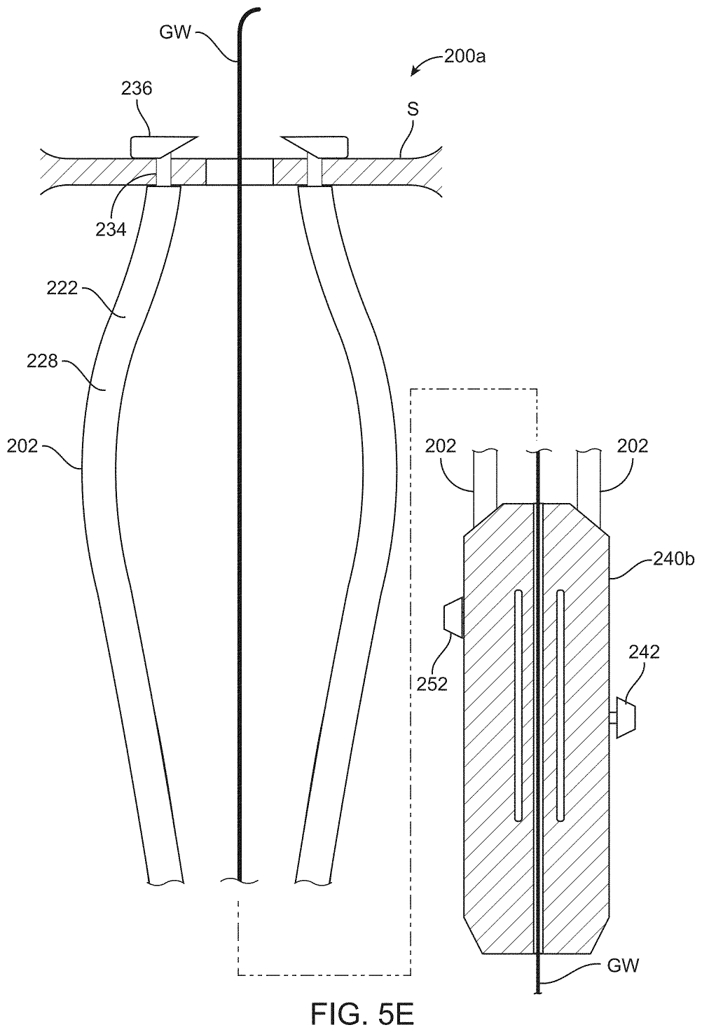

FIGS. 5A-5E show another example of an access and closure system 200a that may be used to pierce the atrial septal wall and close the puncture site after a diagnostic or interventional procedure is completed.

The system and method of using the system are similar to the example shown in FIGS. 4A-4Z2 above but the major difference is the use of protective sheaths to protect the fasteners and help prevent entanglement. The system 200a includes the same disclosure related to the system in FIGS. 4A-4P, and the method begins with and shares the same disclosure as FIGS. 4A-4P with respect to forming the aperture int the septal wall and securing anchors to the septal wall, except that the fasteners are not separated from the handle (as disclosed with respect to FIG. 4P) until later. After the anchors are secured to the septal wall as shown in FIG. 4P, the sheaths may be actuated as shown in FIG. 5A.

FIG. 5A shows further proximal retraction of sliders 250, 244 to proximally retract the pusher 228, needle 222 so they are disposed in sheath 202, and the entire assembly including sheath 202 is retracted proximally. Fastener 234 remains attached to septal wall S via anchor 236 which may be any of the anchors disclosed in FIGS. 4A-4P.

Additionally, sheath 202 is also retracted proximally by actuating slider 252 in the proximal direction. Sheath 202 is coupled with the slider 252, so when slider 252 is advanced or retracted, sheath 202 will move in the corresponding direction. The sheath 202 is retracted so it is removed from open channel 208. Pusher 228, needle 222 are also removed from open channel 208.

FIG. 5B shows further retraction of sliders 250, 244 so that needle 222 and pusher 228 are disposed in sheath 202, and are not visible in FIG. 5B. Also, additional retraction of slider 252 ensures that sheath 202 is removed from the open channel 208 in hub 206. This allows the sheath to bow radially outward away from the elongate shaft 210. The fastener 234 is disposed in the sheath 202 and also bows outward. Bowing is achieved due to the memory in the sheath, or due to a pre-shaped element such as a nitinol wire in the sheath.

FIG. 5C shows that after the sheaths 202 are removed from open channels 208 and bow open, slider 252 is advanced distally moving the sheath 202 distally until it abuts or nearly abuts the septal wall S on the right side of the heart. The pusher 228 and needle 222 remain in their proximally retracted position in sheath 202. The fasteners, here suture filaments 234 are now slidably disposed in the sheath 202. The sheath provides a protective cover to prevent damage to the fasteners 234 that may result as other interventional or diagnostic devices are advanced through the transseptal puncture site. Also, the sheaths may help prevent entanglement of the fasteners 234. And, because the sheaths bow radially outward taking the fasteners 234 with them, this helps move the fasteners out of the way and clears a path for the other interventional and diagnostic devices that may be used in the procedure as they are advanced over the guidewire GW toward the septal puncture.

FIG. 5D shows handle 240 in greater detail. The handle 240 may have two halves, an upper half 240a and a lower half 240b that are releasably coupled together. Sliders 250 and 244 along with actuator 246 may be coupled with the upper half 240a of the handle, while actuators 252 and 242 may be coupled with the lower half 240b of the handle. All components associated with operation of sliders 250, 244 and actuator 246 are also housed in the upper handle half, and all components associated with actuation of actuators 252 and 242 are housed in the bottom handle half. This way, when the top and bottom handle halves are separated from one another, the handle and any shafts or distal components may also be separated into two separate systems and this allows them to be removed from the patient separately as needed.

FIG. 5E shows that after the fasteners 234 are anchored to tissue with anchors 236 and the sheaths 202 are advanced over the fasteners 234, the handles may be separated and the elongate shaft, platform, tapered distal tip may be removed from the patient along with top handle half 240a, as seen in Fig. SF. Only the fasteners 235 covered by sheaths 202 with the needles and pushers in the sheaths are left in the patient and the bottom handle half 240b remains coupled to the fasteners and sheaths.

Once the fasteners are properly positioned and anchored, the septal hole may be dilated using a balloon that is either integral with or separate and discrete from the access and closure system. A diagnostic or interventional procedure may then be performed similarly as described above in FIG. 4S, and then the fasteners may be fixed using any of the devices and methods previously described above, similarly as in FIGS. 4T-4Z2, with the major difference being that the clip, crimping element or knot is loaded over the sheath and the fastener and advanced to the distal end of the sheath and then off the sheath and disposed directly over the fastener where the knot, clip, crimping element is tightened to the fasteners to close or substantially close the septal hole.

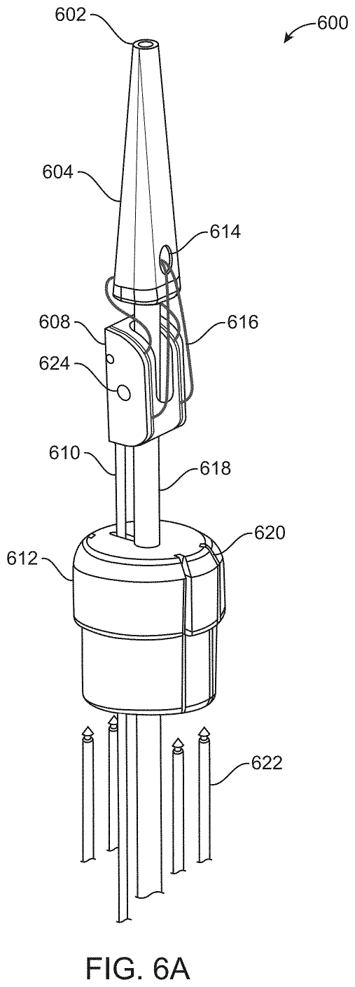

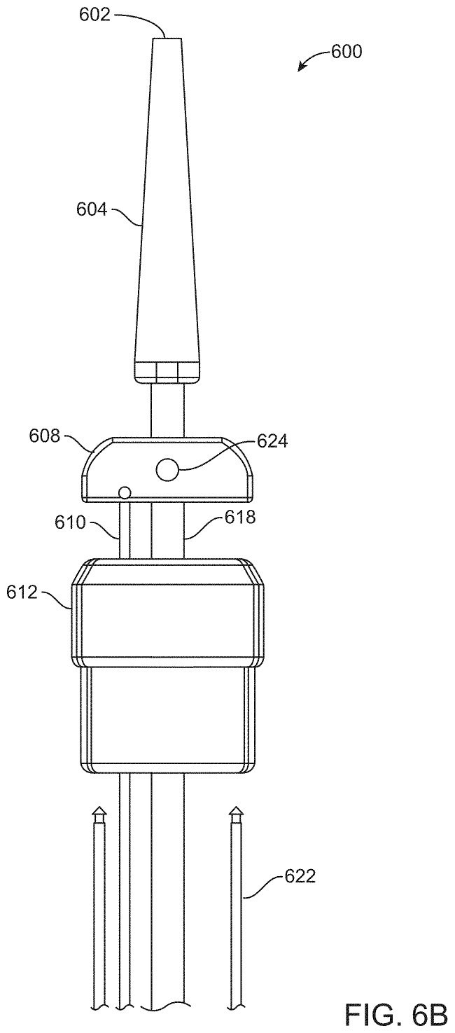

FIG. 6A shows another example of a transseptal access and closure system 600. This system is similar to those previously described above, but instead of the fasteners originating on the right side of the heart and being delivered and anchored to the left side of the heart, the system 600 includes fasteners that are stored on the left side of the heart and then are retracted back into the right side of the heart.

The system 600 includes an elongate shaft 618 having a proximal end and a distal end. A lumen (not shown) extends between the proximal and distal ends. The proximal end of the elongate shaft extends proximally until it is coupled to a handle which will be discussed below. A tapered atraumatic tip 604 is coupled to the distal end of the elongate shaft 618. The tip 604 may include a distal aperture 602 which is fluidly coupled with lumen in the elongate shaft and allows the system to be slidably disposed over a guidewire during delivery.

The tapered tip 604 houses one or more fasteners 616. Here fasteners 616 are sutures. The sutures may be single filament sutures with a free end and an anchor end, or the suture may be looped into a U-shape with two free ends. Additional details about different fasteners 616 is provided below. The fasteners may be removed from the tip 604 via one or more apertures 614 in the tip 604. The free ends of the fasteners are coupled to platform 608 which may be actuated between a collapsed configuration and an expanded configuration. The platform is pivotably coupled 624 to the elongate shaft 610. In this view, the platform is in the collapsed configuration. A pusher rod 610 is also coupled to the platform 608. Actuation of the pusher rod will move the platform between the expanded configuration and the collapsed configuration. The pusher rod 610 extends proximally until it is coupled with a handle as will be disclosed in greater detail below.

A hub 612 is coupled to the elongate shaft 618 near the distal end of the elongate shaft 618. The hub includes open channel 620 which house piercing elements 622. In this example there are four piercing elements 622, although any number may be used. The piercing elements are slidably disposed in the open channels 620 and the pusher rod is also slidably disposed in an aperture in the hub 612. The piercing elements in this example are elongate rods with tissue piercing tips and may also be referred to as piercing shafts. The piercing elements may be needles.

FIG. 6B shows a side view of the access and closure system 600 seen in FIG. 6A but with the fasteners 618 removed and the platform 608 in the expanded configuration. Only two of the four piercing elements 622 are visible in this view and they are retracted proximally. Other aspects of FIG. 6B are the same as in FIG. 6A.

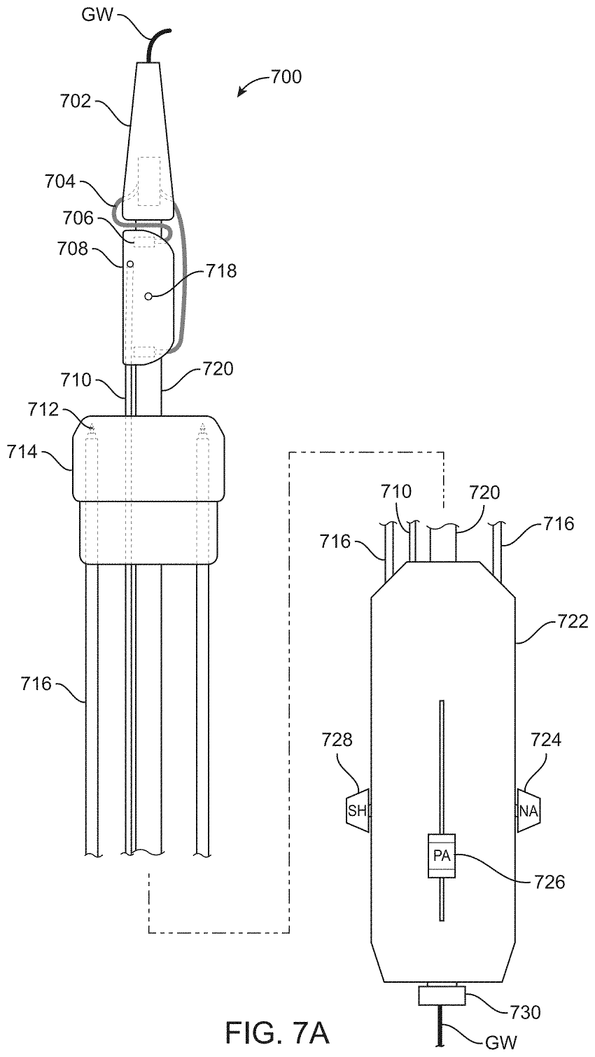

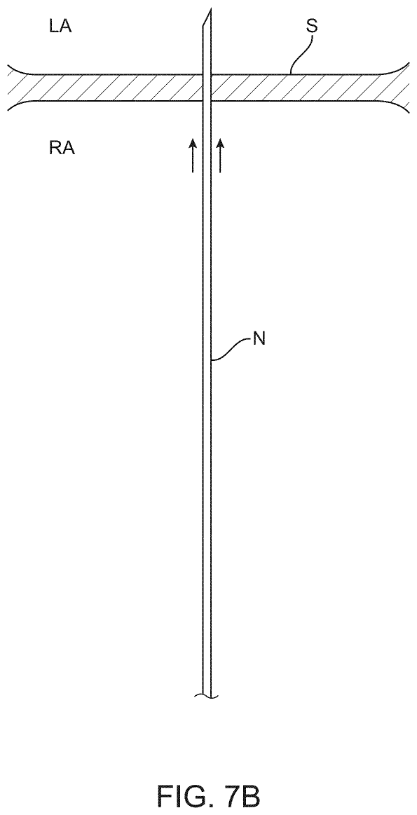

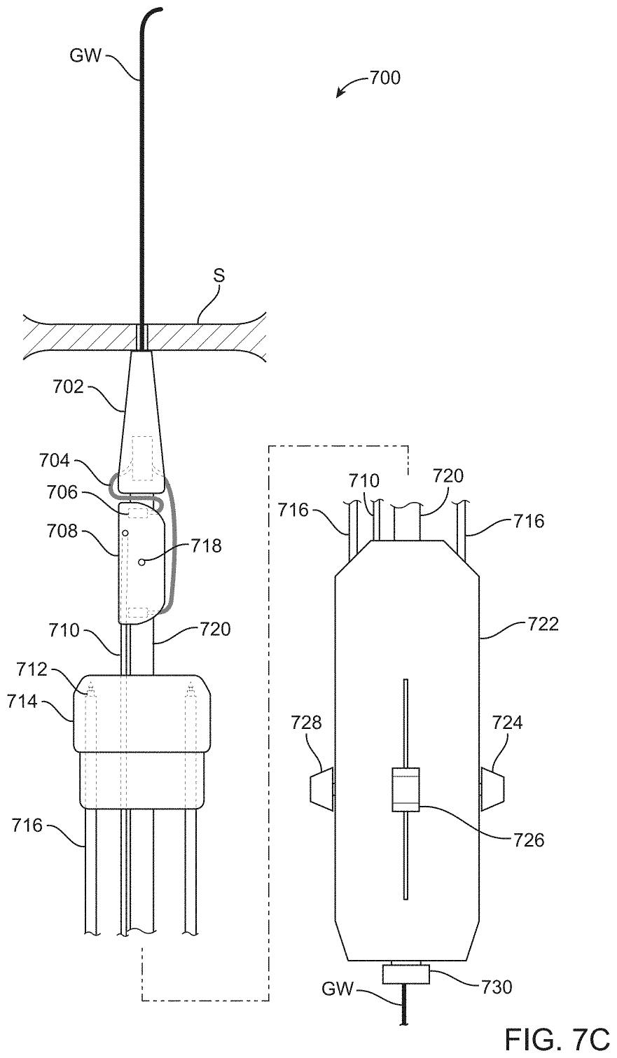

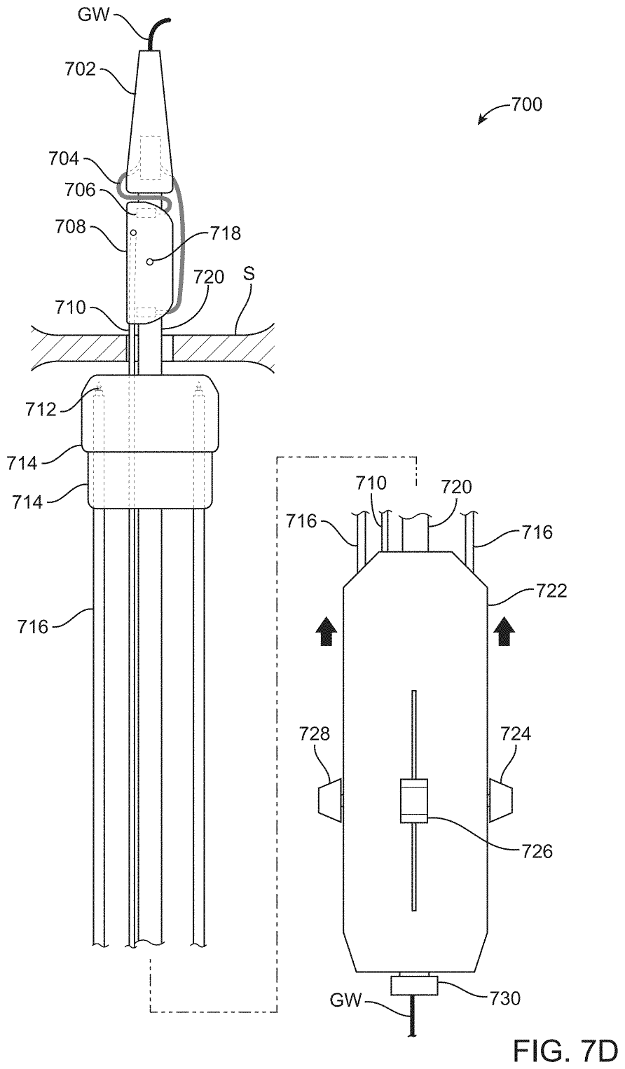

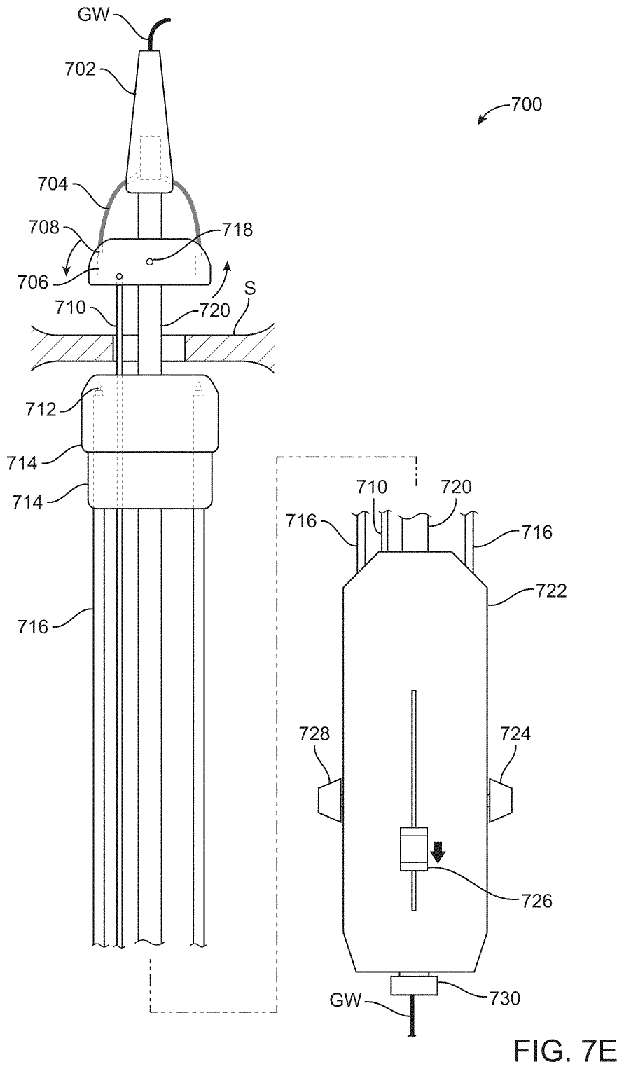

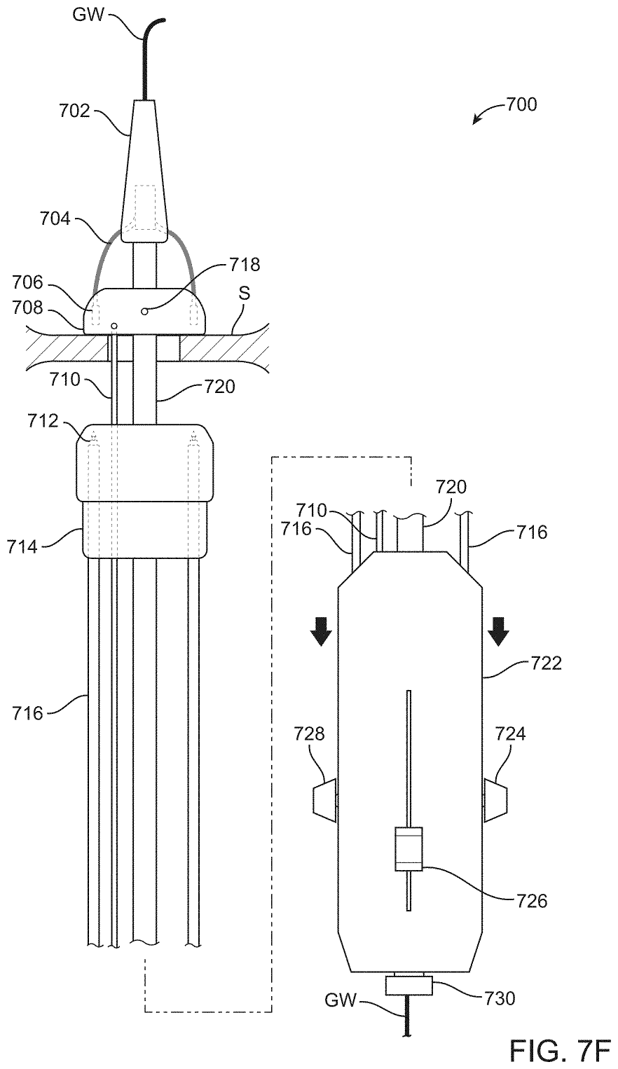

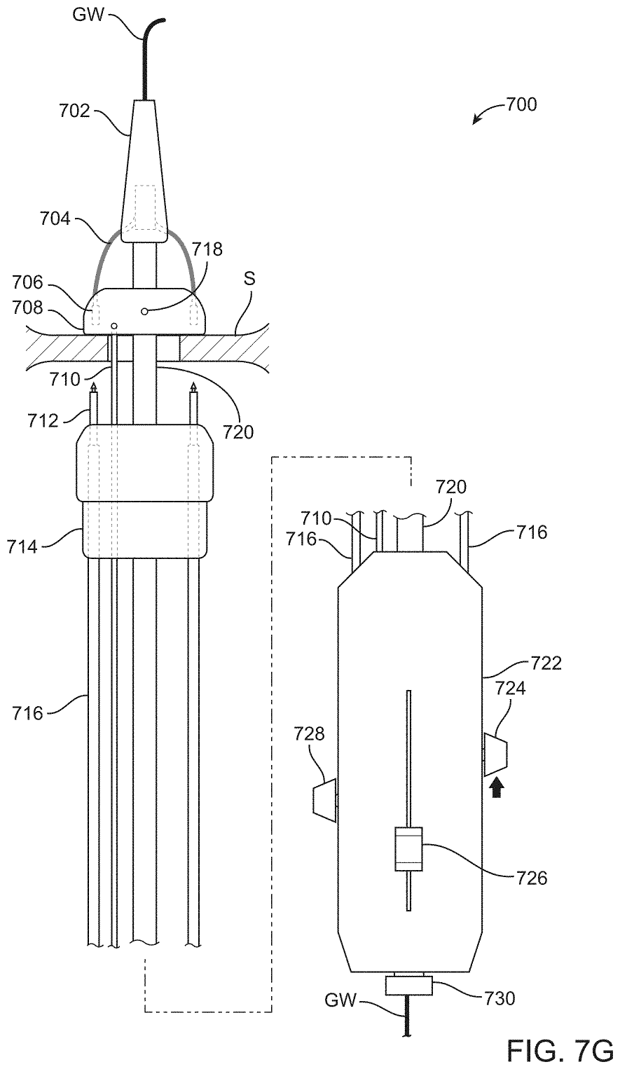

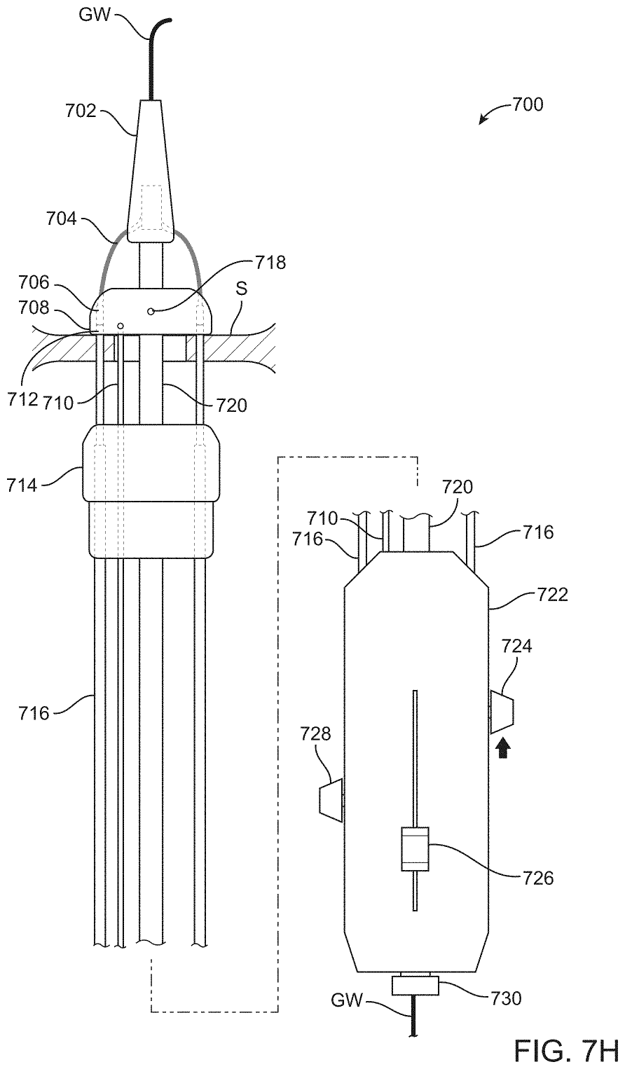

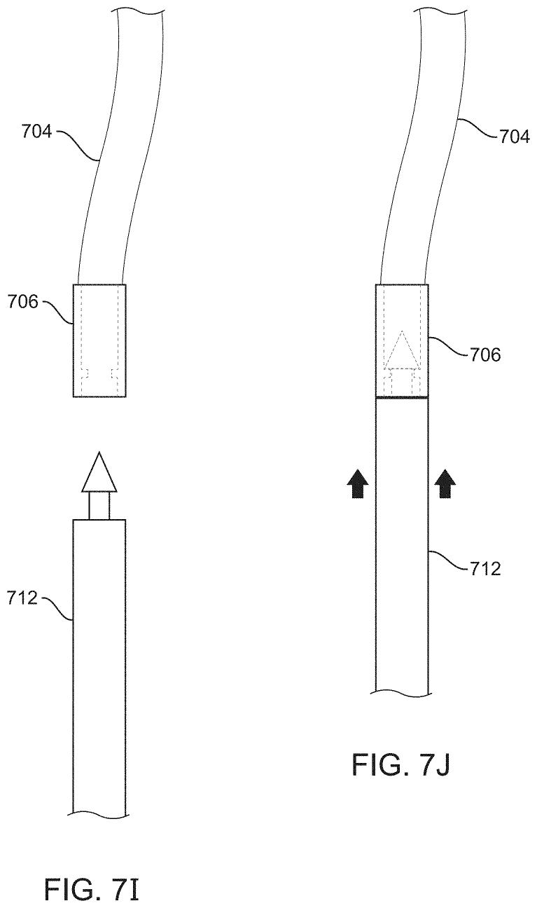

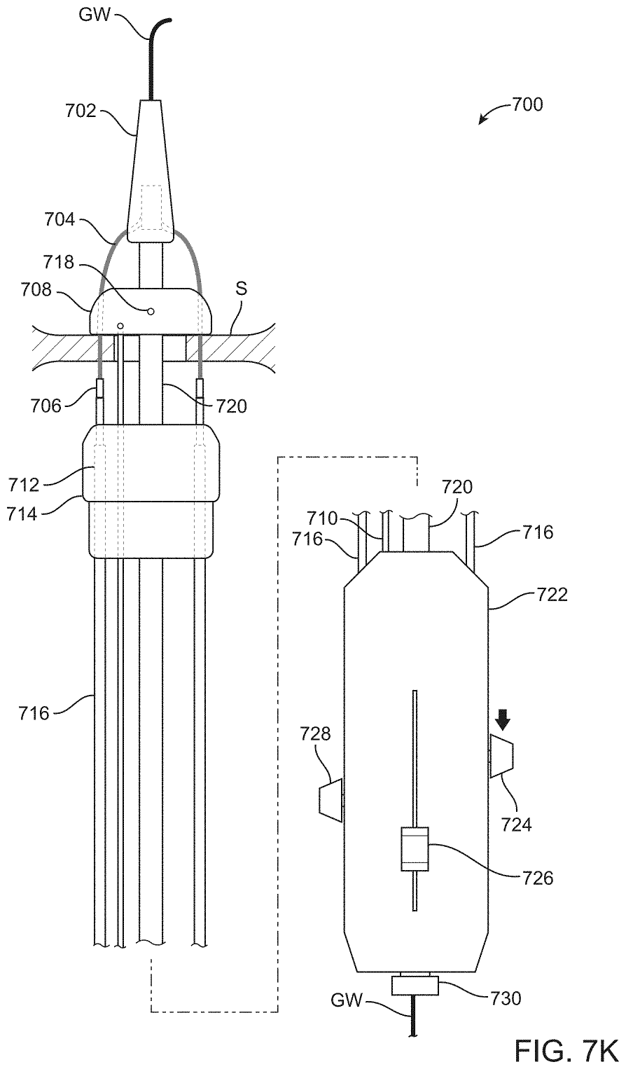

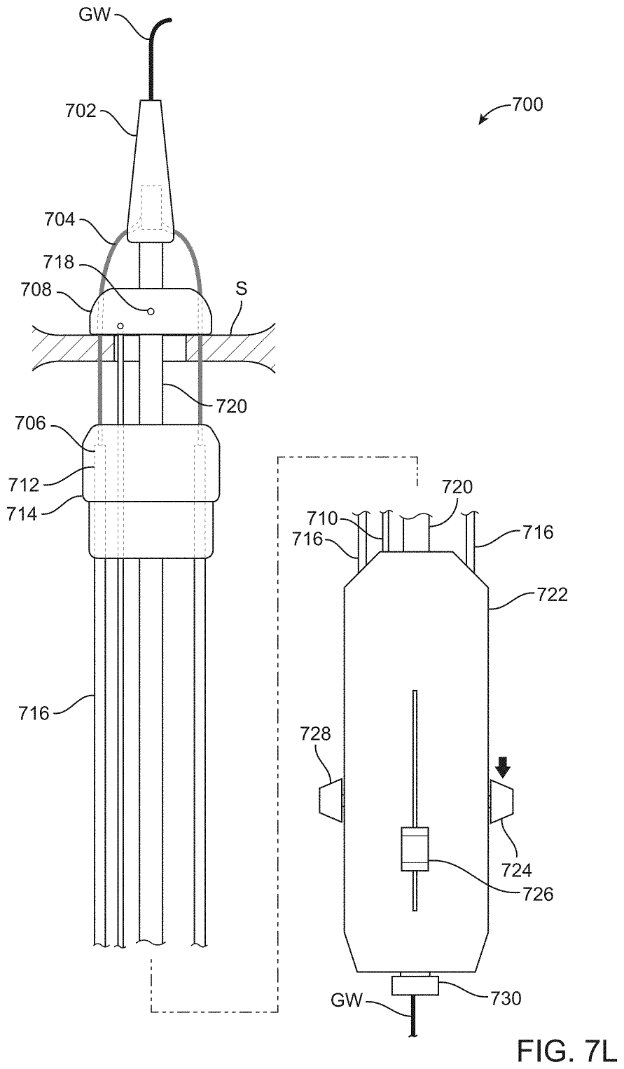

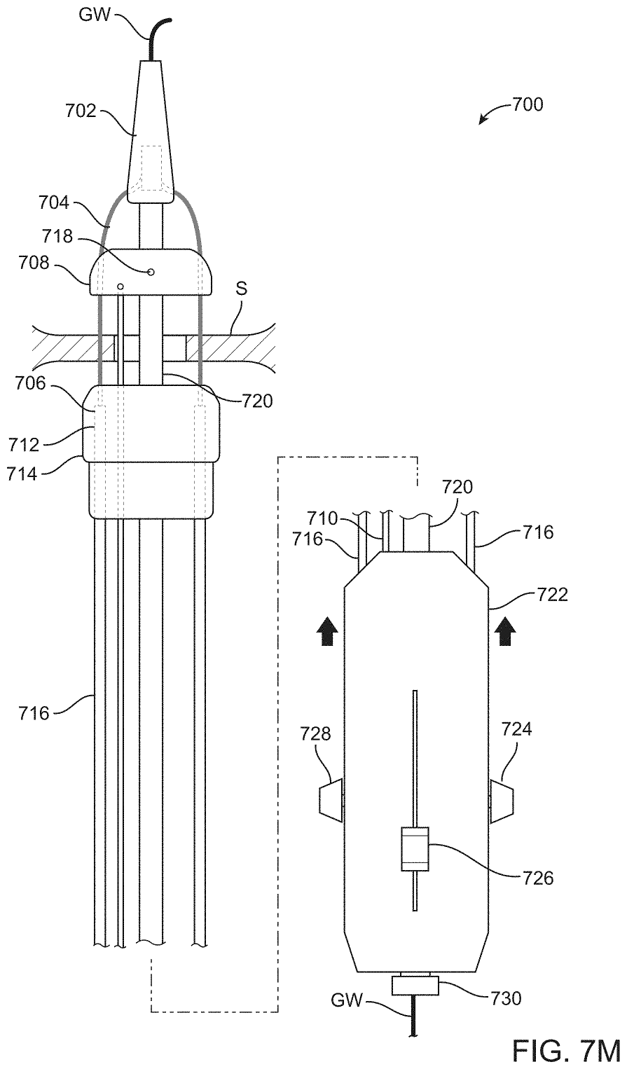

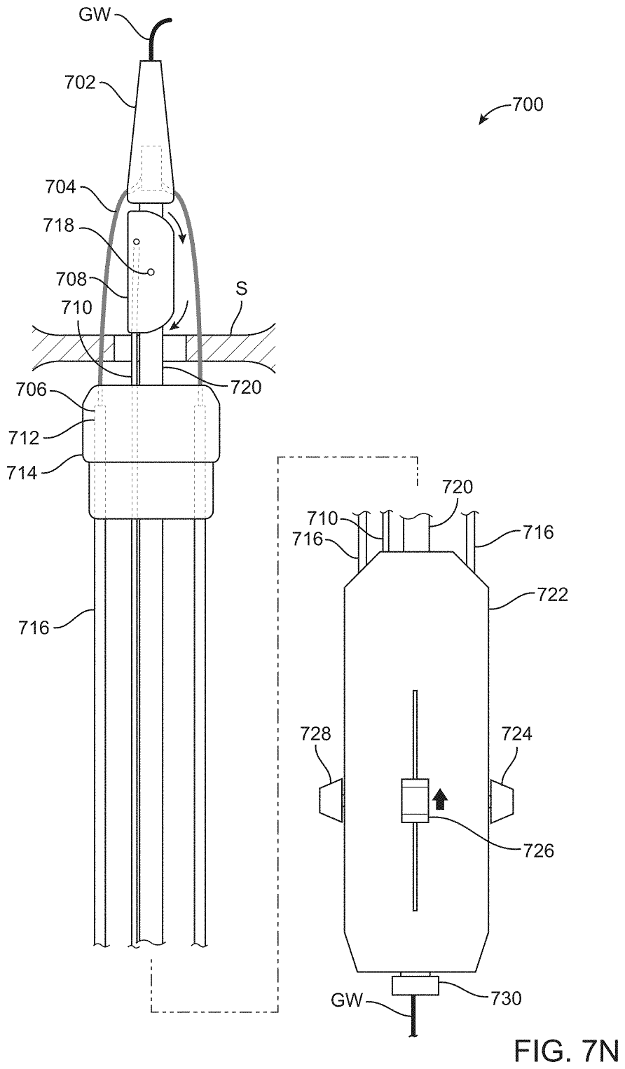

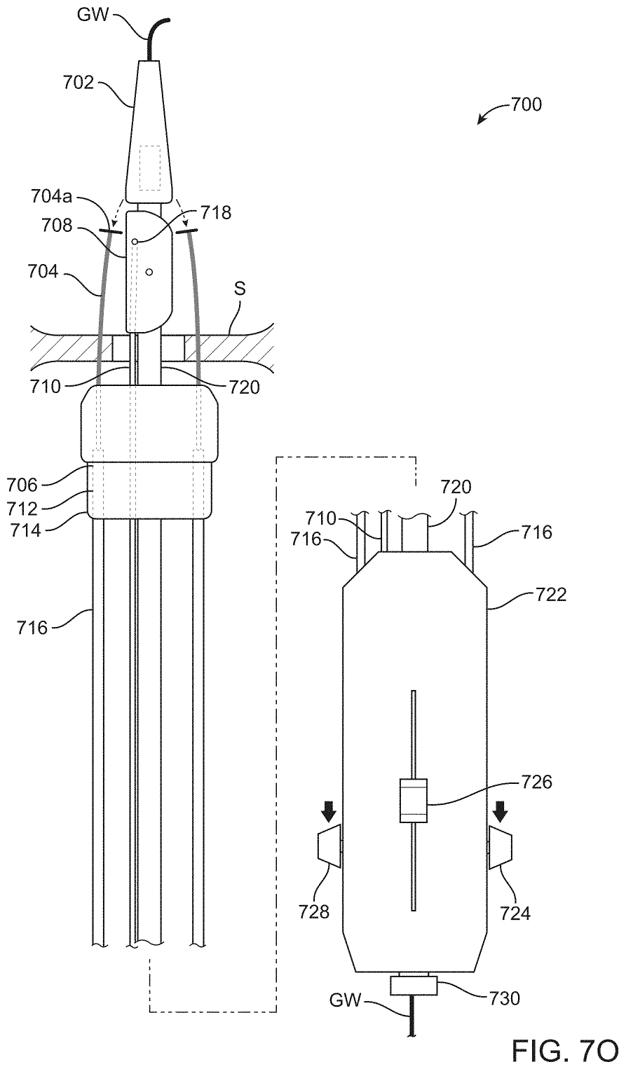

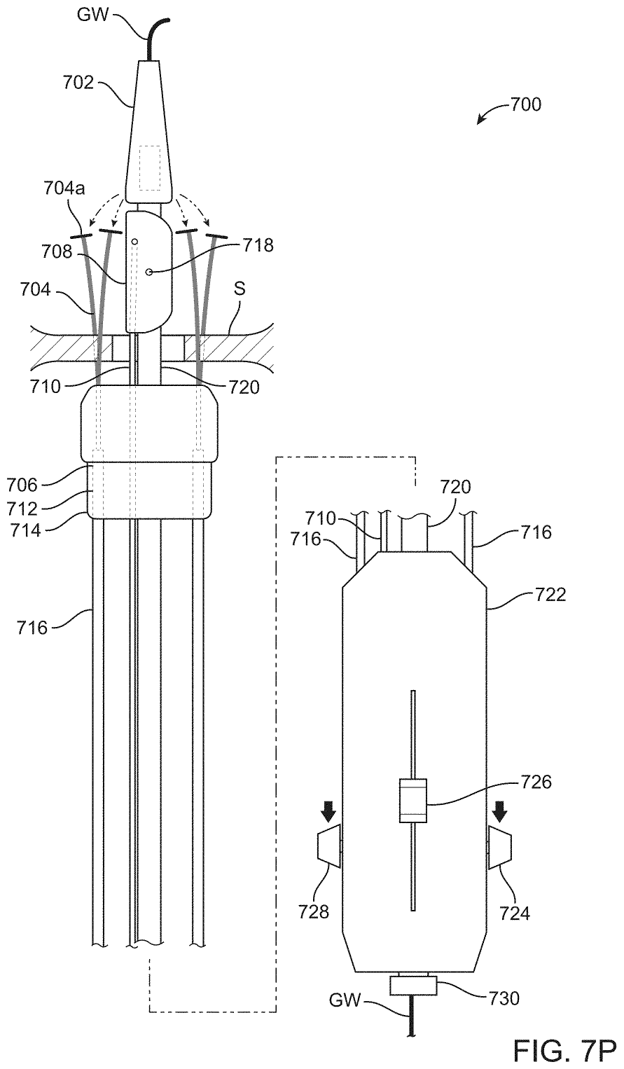

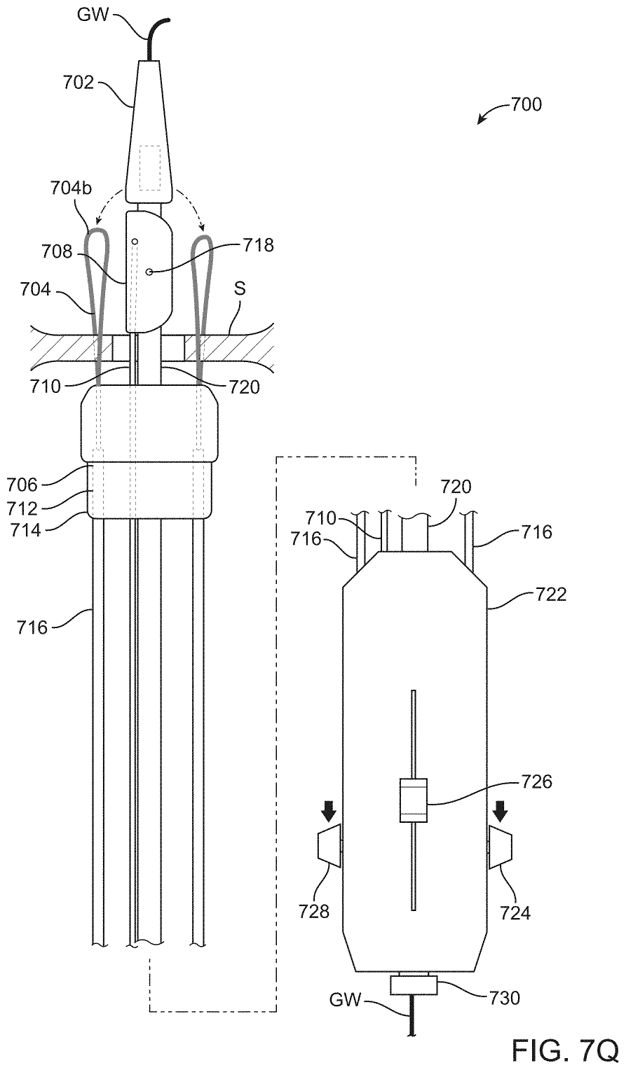

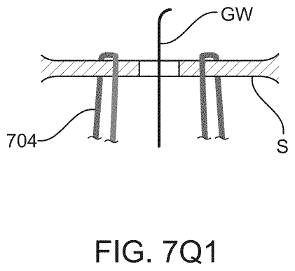

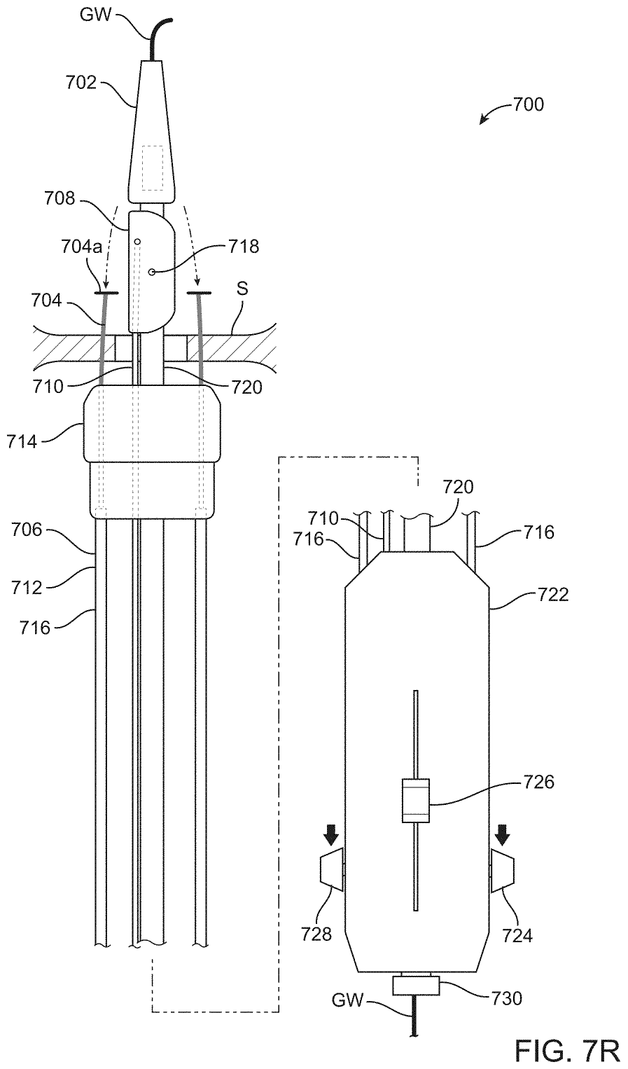

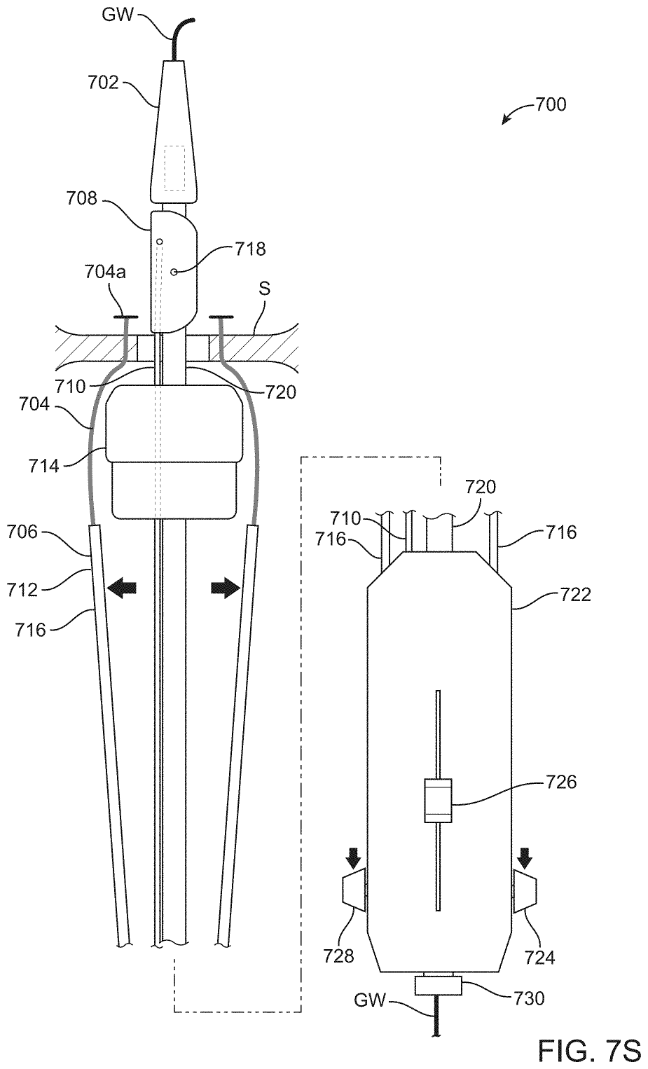

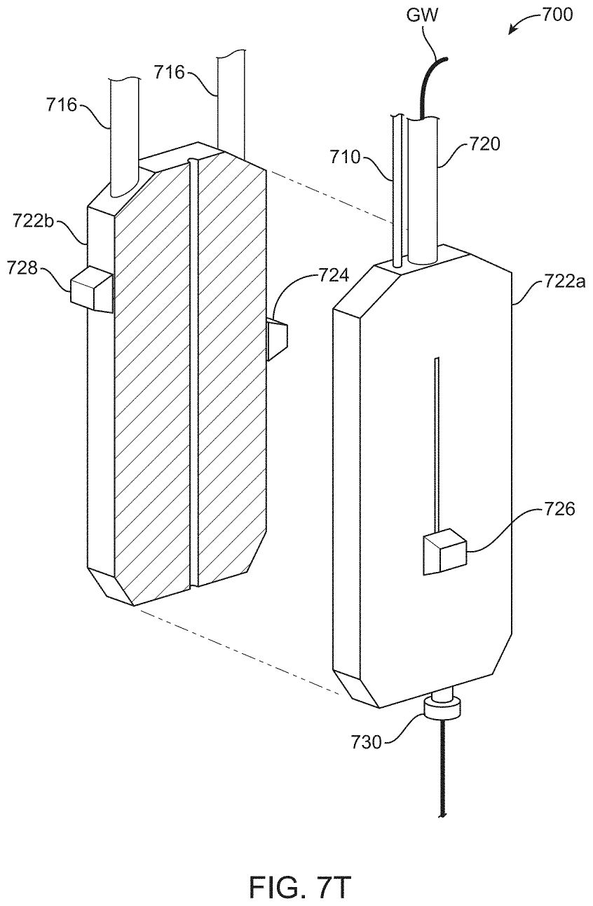

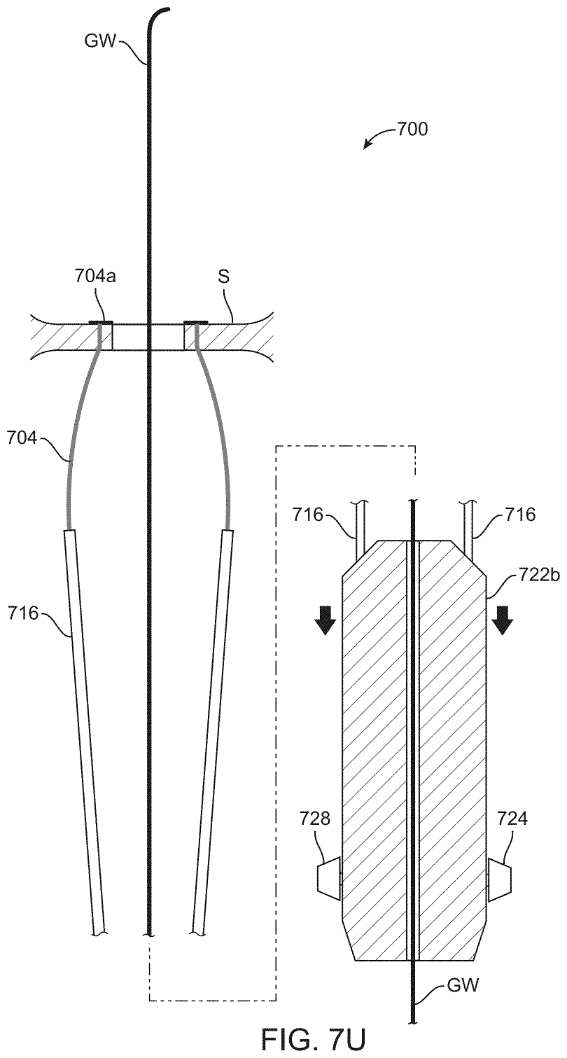

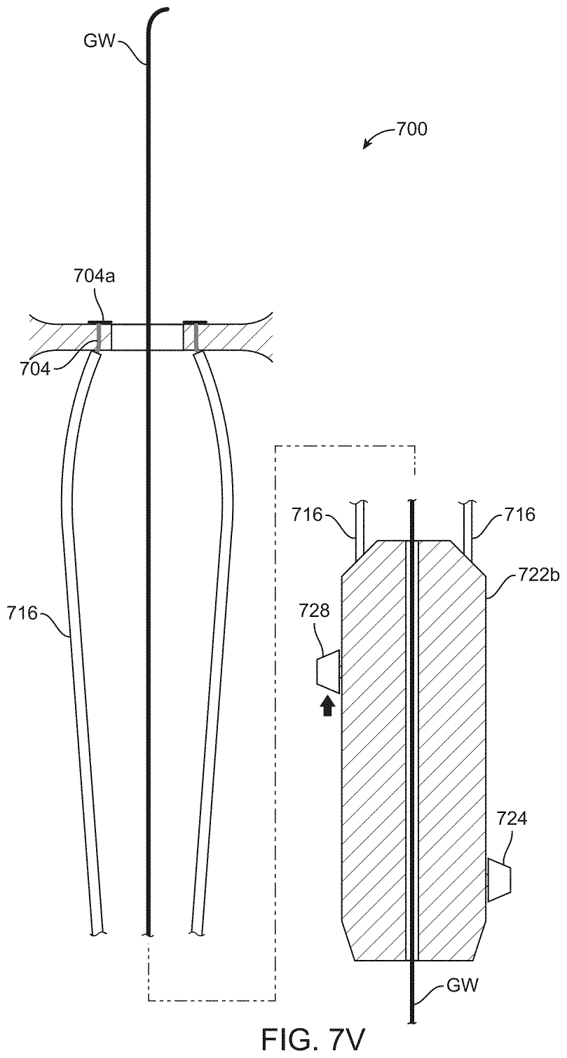

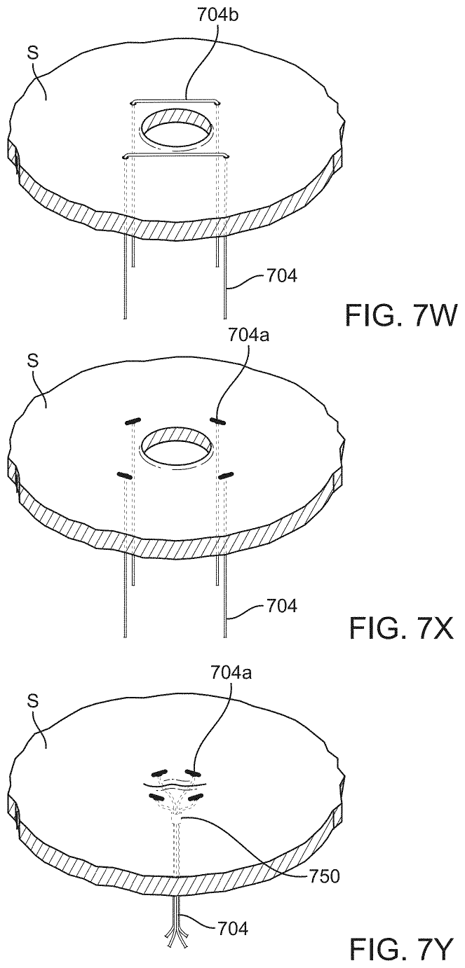

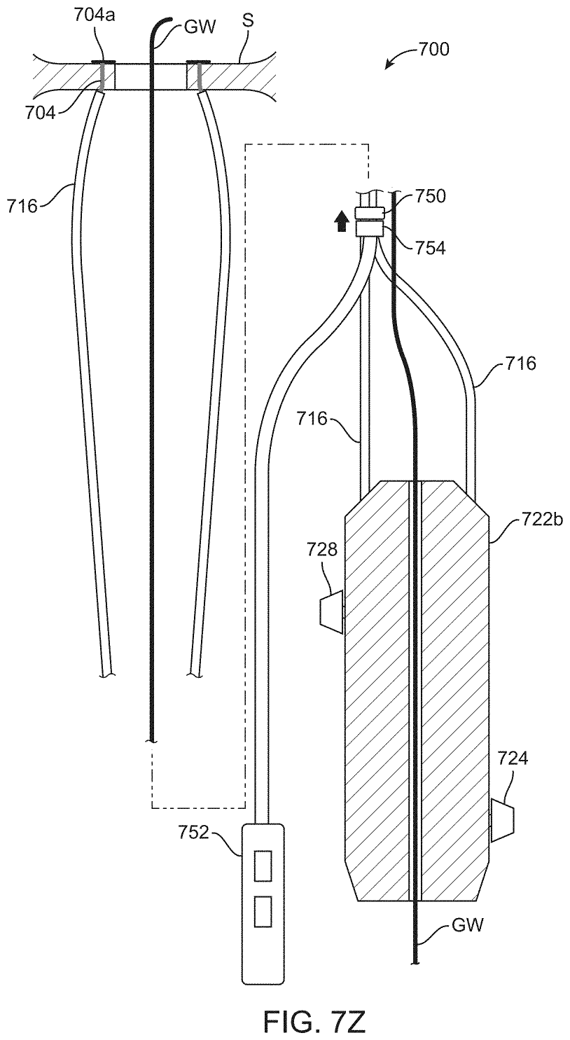

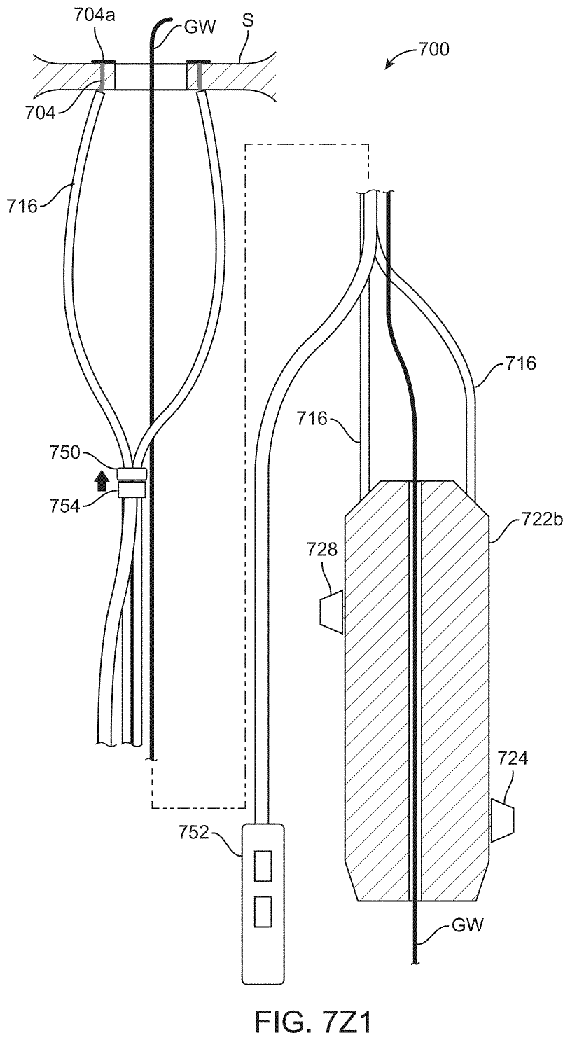

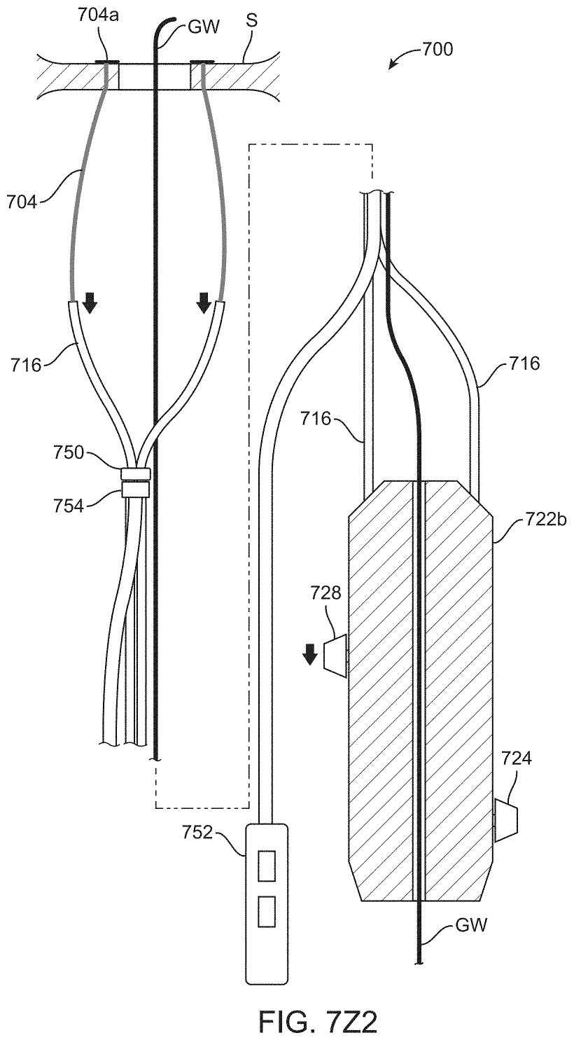

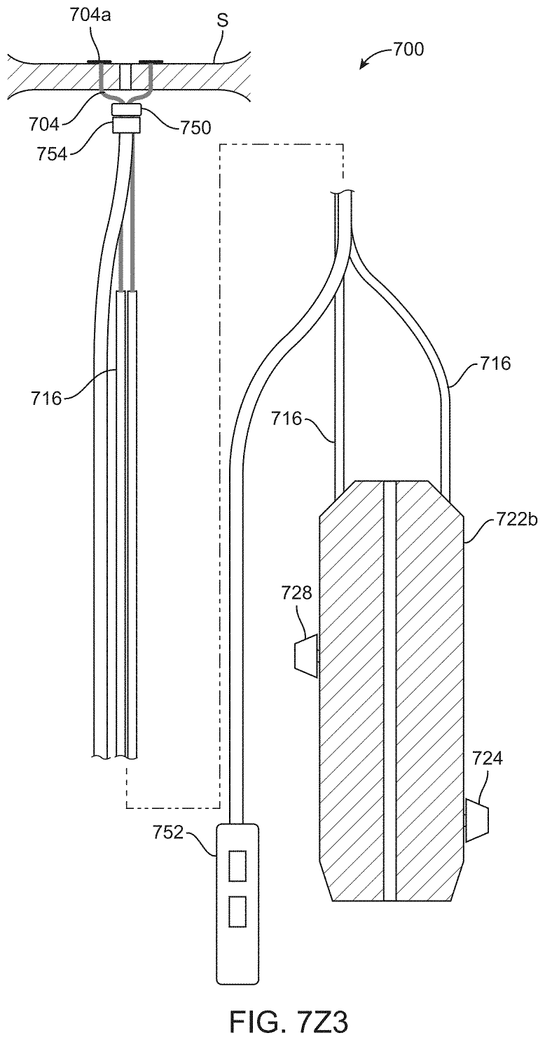

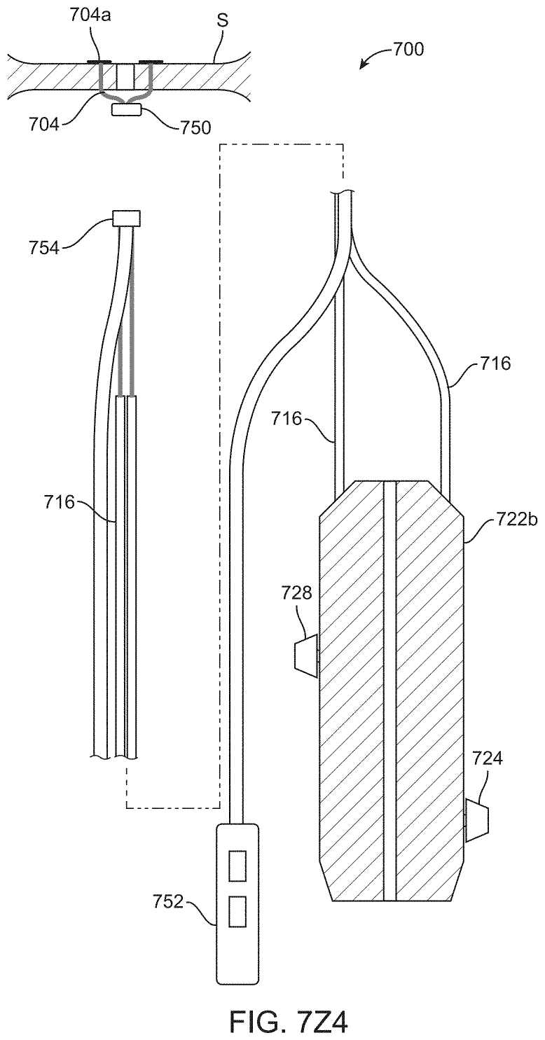

FIGS. 7A-7Z4 illustrate additional details about access and closure system 600 in FIGS. 6A-6B above, along with an example of a method of using the system to provide transseptal access and then septal closure.

FIG. 7A shows a transseptal access and closure system 700 that is substantially the same as described in FIGS. 6A-6B. The system 700 includes an elongate shaft 720 having a proximal and distal end. The proximal end of elongate shaft 720 is coupled to handle 722. The distal end of elongate shaft 720 includes an atraumatic tapered tip 702. A lumen extends from the distal end of the tapered tip 702 through the elongate shaft to hemostasis valve 730 on handle 722. Therefore, a guidewire GW may be inserted through the lumen. One or more fasteners 704 are housed in the tapered tip 702. Here, the fasteners 704 may be a single filament suture, or the suture may be folded on itself to form a U-shaped loop with two free ends. The free end or free ends extend out of tip 702 via an aperture in the tip 702 and they are coupled to the actuatable fastener platform 708. The free end or free ends of the fastener include a coupling element 706 such as a cuff, snap fit, collar or other similar element that allow the free end to be coupled with a piercing element 712 as will be discussed below.

The fastener platform 708 is pivotably coupled 718 to elongate shaft 720 so that the platform may pivot between a collapsed configuration in which the longitudinal axis of the platform is parallel with the elongate shaft, and an expanded configuration where the longitudinal axis of the platform is orthogonal or transverse to the longitudinal axis of the elongate shaft. In the expanded configuration the platform abuts tissue and supports the tissue so that the piercing elements can pierce through tissue without the tissue bowing away from the piercing elements. In FIG. 7A, the platform is shown in the collapsed configuration.

A pusher rod 710 is coupled to the platform and extends proximally and is coupled with handle 722. Actuation of the pusher rod moves the platform between the expanded and collapsed configurations.

A hub 714 is coupled with elongate shaft 720 adjacent the distal end of the elongate shaft. The hub has open channels for slidably receiving the piercing elements 712 (sometimes also referred to herein as a penetrating shaft) which include a tissue piercing tip that can pierce tissue and also attach to the coupling elements 706 on the free ends of the fasteners 704. The piercing elements 712 are also slidably disposed in sheaths 716 which will receive and protect the fasteners later in the method. The piercing elements 712 are housed in a retracted configuration in the hub 714 and in sheath 716 during delivery to prevent unwanted piercing, and when desired the piercing elements may be actuated into an extended configuration where they pass through the septal wall and engage the coupling elements in the fastener platform.

The sheaths 716, pusher rod 710, and elongate shaft 720 are coupled to handle 722 which include actuators 724, 726, 728. The actuators may be any actuator such as knobs, wheels, levers, sliders, etc. but in this example the actuators are sliders. Actuator 724 is a slider which controls actuation of the piercing elements 712. Actuator 726 is also a slider and controls movement of the pusher rod 710. Actuator 728 is a slider and control movement of the sheaths 716. The handle may have two halves which may be separated from one another as will be described below. The proximal end of the handle 722 may include a hemostasis valve 730 for controlling fluid flow (e.g. blood) from out the proximal end of the lumen. Examples of hemostasis valves include duck bills valves or Tuohy-Borst valves which may also be used to tighten down on a guidewire or other object inserted in the proximal end of the handle to prevent unwanted axial movement. The guidewire GW exits the proximal end of the handle 722 via the hemostasis valve 730.

FIG. 7B shows the initial step in a transseptal access and closure procedure that may be performed using the system in FIG. 7A. Here, a tissue piercing needle N is advanced from the right atrium RA to the left atrium LA by piercing through the atrial septum S. The needle N maybe a Brockenbrough needle or other needle used by cardiologists. The needle may be advanced over a guidewire and once the septal wall is pierced, the guidewire may be advanced across the septal wall and the needle retracted proximally. Here the piercing needle is a separate device from the transseptal access and closure device, while in other examples below the piercing needle is integral with the system.

FIG. 7C shows that after the atrial septum has been pierced and a guide wire GW advanced across the septum 5, the transseptal access and closure system 700 may be loaded over the guidewire GW and advanced distally. The distal tip 702 is advanced until it is adjacent the hole in the septum S on the right side of the heart, with the rest of the working end of system 700 still in the right heart.

FIG. 7D shows the entire system 700 advanced distally as indicated by the arrows so that the tapered distal tip 702 is advanced through the hole in the septum S. Distal advancement is performed by the operator pushing the handle 722 distally so that the entire system moves distally. The tapered tip is advanced distally enough so that the apertures 614 (best seen in FIG. 6A) are on the left side of the heart in the left atrium. Additionally, the fastener platform 708 is also now disposed on the left side of the heart along with the fasteners 704 that are housed in the tip 702 with free ends coupled to the fastener platform 708. The fastener platform 708 is still in the collapsed configuration so that it can fit through the hole in the septal wall S. In the collapsed configuration, the fastener platform 708 has its longitudinal axis parallel with the longitudinal axis of the elongate shaft 720.

FIG. 7E shows that after the fastener platform 708 is disposed on the left side of the heart, the fastener platform 708 is actuated from the collapsed configuration to the expanded configuration. Here, the platform is pivoted about pivot point 718 where the platform is pivotably coupled to elongate shaft 720 so that the longitudinal axis of the platform 708 is now orthogonal or transverse to the longitudinal axis of the elongate shaft. Because the length of the platform is longer than the diameter of the hole pierced through the septum S, the platform will span the length of the hole and abut tissue surrounding the pierced hole and cannot be pulled back through the hole. The platform is actuated by retracting actuator 726 proximally as indicated by the arrow thereby also retracting push rod 710 proximally and causing the platform to pivot into the expanded configuration. The push rod 710 is operably coupled to the actuator 726 which in this example is a slider, although the actuator may be other mechanisms such as knobs, wheels, sliders, levers, etc. The fasteners 704 are also partially pulled out of tip 702 as the platform deploys.

FIG. 7F shows once the platform 708 has been deployed into the expanded configuration, the entire system 700 is retracted proximally as indicated by the arrows so that the platform abuts the tissue surrounding the hole pierced in the septal wall S. The platform then provides support to the tissue surrounding the septal hole as the piercing elements are advanced through the tissue. The system may be retracted proximally by pulling the handle 722 proximally since the handle is operably coupled to all of the shafts and piercing elements.

FIG. 7G shows distal advancement of piercing elements 712 toward the septal wall S. The piercing elements here are needles with sharp tips. They are advanced by actuating actuator 724. Actuator 724 in this example is a slider but could be any other actuator such as a knob, wheel, lever, etc. The slider is operably coupled with the piercing elements 712. The piercing elements are advanced distally, so the sharp point is exposed from both the hub 714 and sheath 716. The sheath 716 remains disposed in hub 714.

FIG. 7H shows further distal actuation of slider 724 as indicated by the arrow advances the piercing elements 712 through the septal wall and into engagement with the apertures in the fastener platform 708 where the sharp tip engages the coupling element 706 attached to the free ends of the fasteners 704. The fastener platform supports the tissue surrounding the piercing and facilitates the piercing element passing through the tissue. This locks the piercing element with the fastener as will be more clearly illustrated in the following figures.

FIGS. 7I-7J show an example of how the piercing elements 712 may be coupled with the coupling element 706 on the free ends of the fasteners 704. In FIG. 7I, the piercing tip of the piercing element 712 is advanced toward the coupling element 706 until the two engage one another as seen in FIG. 7J. The piercing tip then may form a snap fit with the coupling element joining the two together. An enlarged head region on the piercing tip may snap into a cooperating recess in the coupling element.

FIG. 7K shows that after the piercing elements have engaged the coupling element on the fasteners, actuator 724 may be retracted proximally to retract the piercing elements 712 proximally so they are removed from the left side of the heart and removed from the septal wall S and are now disposed on the right side of the heart. As the piercing elements 712 are retracted proximally, they will also pull the free ends of fasteners 708 with them so the fastener is also retracted proximally across the septal wall into the right side of the heart. The remainder of the fastener is still housed in the tip 702.

FIG. 7L shows further proximal retraction of slider 724 which draws the piercing elements 712 into sheath 716 which is disposed in the open channels of the hub 714. The fastener 704 and coupling element 706 is also drawn into the sheath 716. The tip of the piercing element 712 and the free end of the fastener is therefore now protected by sheath 716.

FIG. 7M shows that distal advancement of the entire system 700 by pushing handle 722 distally disengages the fastener platform from the septal wall S. This allows the fastener platform to be actuated from the expanded configuration back into a collapsed configuration.

FIG. 7N shows fastener platform 708 actuated from the expanded configuration to the collapsed configuration which is a lower profile to allow retraction of the system from the left side of the heart hack to the right side of the heart via the hole pierced in the septal wall S. Here, actuator 726 is slidably advanced distally to push pusher 710 distally causing fastener platform 708 to pivot about pivot point 718 into the collapsed configuration where the longitudinal axis of the platform is substantially parallel with the longitudinal axis of the elongate shaft 720 so that the width of the platform is small enough to fit back though the hole in the septal wall S.

In FIG. 7O, sliders 728 and 724 are retracted proximally as indicated by the arrows. Slider 728 is operatively coupled with the sheaths 716 therefore as slider 728 is retracted, sheaths 716 are also retracted proximally. Slider 724 is operatively coupled with piercing elements 712 which are disposed in the sheaths 716, therefore both the sheath 716 and the piercing elements 712 are retracted proximally. Additionally, piercing element 712 is coupled to coupling element 706 on the free end of the fastener element 704, therefore the fastener element 704 is also retracted proximally, until the entire fastener 704 is pulled out of the tip 702 and the anchor end 704a is free of the tip. Here, the anchor end is a T-tag having a crossbar that is transverse or orthogonal to the longitudinal axis of the fastener 704. Other anchors that have material on the end of the fastener that prevent the fastener from being pulled through the tissue may be used here or in any fastener example may be referred to as a pledget. FIG. 7O also shows two fasteners 704. The crossbar prevents the anchor end from being pulled through the tissue. Other configurations of the anchor end are possible and will be disclosed in greater detail below.

FIG. 7P shows substantially the same step as FIG. 7O with the major difference being the number of fasteners 704. Here, there are four fasteners 704 which are originally stored in the tip 702 and then coupled with four cooperating piercing elements 712 via the coupling element 712. Thus, as the sheaths 716 and the piercing elements 712 are proximally retracted when sliders 724, 728 are retraced proximally, four individual fasteners, each a single filament, are pulled out of tip 702 to expose four anchor ends with T-tags 704a. Other aspects of FIG. 7P are generally the same as in FIG. 7O. Four fasteners or two fasteners may be used and this is not intended to be limiting, and any number of fasteners may be used.

FIG. 7Q shows substantially the same step as FIG. 7O or 7P with the major difference being the number of fasteners 704. Here, there are two fasteners 704 that are each a strand of suture looped on itself with the two free ends coupled to the piercing elements 712 via coupling elements 706. The free ends are pulled into sheath 716 via separate piercing sites in the septal wall S. The looped or U-shaped portion 704b is pulled out of tip 702 and is unable to pass through the tissue and therefore forms the anchor portion of the fastener. Thus, in this example, there are two fasteners with four strands of suture that extend across the septal wall into the right atrium. Other aspects of FIG. 7Q are generally the same as in FIG. 7O or 7P. The fasteners in FIG. 7Q may be used in conjunction with or as a substitute for the fasteners in FIG. 7O or 7P.

FIG. 7Q1 shows the fasteners 704 anchored to the septum with the free ends passing through the septal wall and extending into the right atrium while the U-shaped portion of the looped fastener remains on the left side of the heart anchoring the fastener. Here, four needles or piercing elements are used to capture the free ends and retract them proximally through the septal wall into the right side of the heart.

FIG. 7R follows after FIG. 7O and assumes that two fasteners 704 are used and not the variations shown in FIGS. 7P-7Q1. Here, further retraction of both sliders 728, 724 continue to proximally retract the sheaths 716 and the piercing elements 712 thereby further drawing fasteners 704 proximally. The anchor ends 704a, here T-tags are also drawn further toward the left side of the septal wall S. The sheaths 716 still disposed in the open channels of hub 714.

FIG. 7S shows that even further proximal retraction of sliders 728, 724 eventually pulls the sheaths 716 out of the open channels in hub 714 thereby allowing them the bow outward to clear a path for an intervention or diagnostic device. The sheaths may have memory imparted in them to cause the bowing, or there may be a spring coupled to the sheath such as a nitinol wire which causes the bowing. The fasteners 704 and the piercing elements 712 also bow outward along with the sheaths 716.

FIG. 7T shows the handle 722 in more detail. In this example, the handle 722 may have an upper handle portion 722a and a lower handle portion 722b that are releasably coupled together. They may be clipped, snap fit, or otherwise coupled together and the two halves may be easily released from one another. The top half 722a may include the actuator that controls the pusher 710 and also elongate shaft 720 is coupled to the upper handle half 722a along with the platform 708, hub 714, and tip 702. The bottom handle half 722b includes actuators 724, 728 which are coupled to the piercing elements 712 and the sheaths 716. Therefore, when the handle is split into two halves, the upper handle half may be retracted proximally removing the elongate shaft 720, pusher rod 710, platform 708, hub 714, and tip 702 from the patient, leaving only the fasteners 704 coupled to the piercing elements 712 via coupling elements 706 disposed in sheaths 716 in the body, along with the guidewire GW.

FIG. 7U shows that after the handle 722 has been split into its two halves and the top half 722a retracted to remove the elongate shaft 720 and everything coupled to it from the patient's body, only the fasteners 704 disposed in sheaths 716 are left behind. The fasteners 704 are anchored to the left side of the septal wall with anchors 704a, which in this example are the T-tags but they may be any of the others disclosed herein. The fasteners are still coupled to the piercing elements 712 via coupling elements 706, which are disposed in sheath 716. Now, fasteners are secured to the septal wall and a path is cleared for introduction of an interventional device or diagnostic device over guidewire GW which may be left in place. Proximal retraction of bottom handle half 722b, as indicated by the arrows, ensures that the anchors are firmly apposed with tissue surrounding the hole pierced in the septal wall S.

FIG. 7V shows distal advancement of slider 728 in order to advance sheaths 716 distally over the fastener 704 until the distal end of the sheath 716 abuts the septal wall S or nearly abuts the septal wall S. The sheath provides an outer protective covering for the fasteners 704 to prevent damage or minimize the possibility of entanglement.

FIGS. 7W-7Y show different fasteners 704 from the left side of the heart disposed in tissue surrounding the septal puncture in the atrial septum. This view presents the fasteners in FIG. 7V but with the sheath 716 removed for convenience in viewing.

FIG. 7W shows the fastener 704 that is formed from a single filament of suture that is looped to form a U-shape. The 1J-shape forms the anchor 704b which prevents the fastener from pulling out of the left side of the heart and is the same as in FIG. 7Q. Each fastener therefore has two strands of suture that extend back into the right side of the heart and in this example there are two fasteners, with a total of four strands.

FIG. 7X shows the fastener 704 that is formed from a single filament of suture with a T-tag 704a which anchors the fastener to the left side of the heart, and is the same as in FIG. 7P where there are four fasteners each having a T-tag and four strands extend into the right side of the heart.

FIG. 7Y shows closure of the septal piercing when tension is applied to the fasteners 740 to appose tissue around the perimeter of the piercing. A securing element 750 such as a clip or crimping element is then applied to the fasteners to maintain tension in the fasteners and keep the pierced hole closed or substantially closed. This example shows the use of four fasteners with T-tags as in FIG. 7X, but could easily be substituted with any of the other examples of fasteners disclosed herein. Additional information about the securing element is disclosed below.

Once the fasteners 704 are properly deployed and the path to the septal hole is free, the hole may be dilated with an expandable member such as a balloon as discussed above in relation to FIGS. 4R-4S, and then an interventional or diagnostic procedure such as that shown previously in FIG. 4S may be performed. After the interventional procedure is completed, the fasteners maybe secured to close the septal puncture.

FIG. 7Z shows that after fasteners have been properly deployed and anchored and the sheath advanced over the fasteners, a securing device 752 having a securing element 750 and a cutting element 754 may be advanced over the sheaths 716. The securing element 750 may be a clip, a crimping element, or any other element that can be used to secure the fasteners. The cutting element 754 may be a cutting blade or other element for severing the fasteners to a desired length. Since the securing device 752 is advanced over the sheaths 716, the securing device 752 need not be advanced over the guidewire GW.

FIG. 7Z1 shows further distal advancement of securing instrument 752 so that the cutting element 754 pushes the securing element 750 distally along the sheaths 716 toward the septal wall S.

FIG. 7Z2 shows that after the securing element 750 has been advanced distally into the patient and is adjacent the septal wall 5, slider 728 may be actuated proximally to draw sheaths 716 proximally and away from the septal wall S exposing fasteners 704.

FIG. 7Z3 shows the guidewire GW removed from the septal piercing in the septal wall S and further distal advancement of the securing element 750 by advancing the securing device 752 distally thereby pushing the securing element 750 distally over the sheath 716 and then off the sheath 716 onto the fastener 704 until it is engaged with or nearly engaged with the septal wall S. Tension in the fasteners 704 is applied in order to draw tissue surrounding the pierced hole together to close or nearly close the septal puncture.

FIG. 7Z4 shows that after the fasteners 704 have been adequately tensioned and the pierced hole in septum S is closed or substantially closed, the securing element 750 may be applied to the fasteners 704 to hold them in position. For example, an actuator such as a slider, lever, or knob on a handle on the securing device 752 may be actuated to close the securing device 750 to the fasteners to hold them in position and maintain the septal piercing closed or substantially closed. Clamping element 754 may be a crimper or other element for closing a clip around the fasteners. Additionally, clamping element 754 may include a cutting element that may be controlled by actuating an actuator on the handle of securing device 752 to cut the excess fasteners to a desired length so the excess may be removed from the patient. The securing device 752 and excess fasteners may then be removed from the patient along with the sheaths 716.

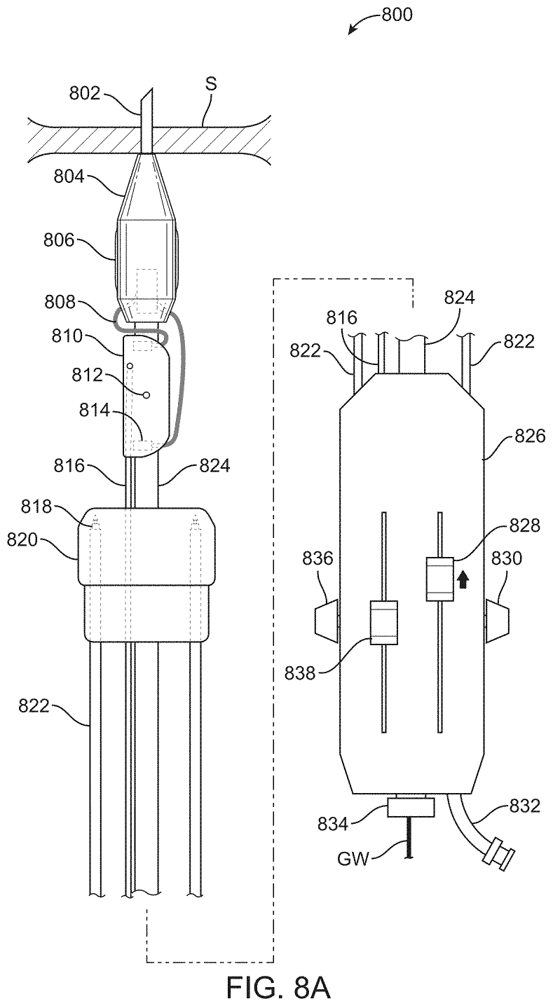

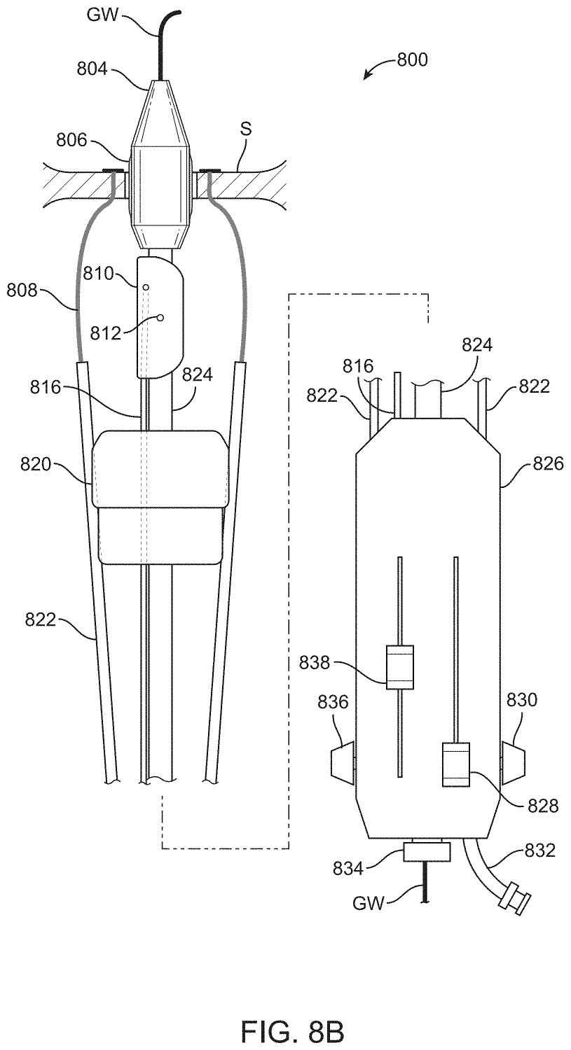

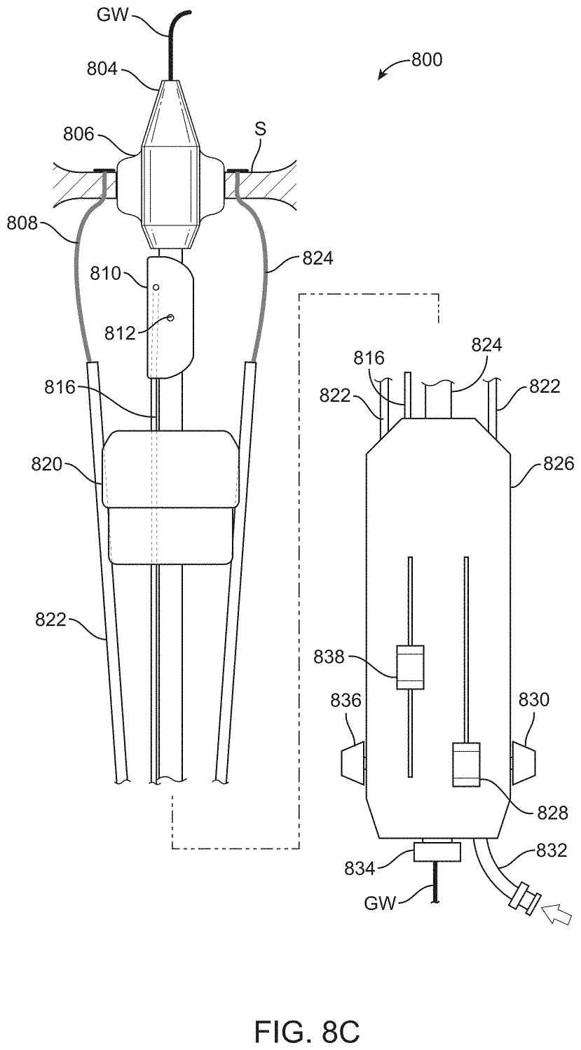

FIGS. 8A-8C illustrate another example of an atrial septal access and closure system 800.

In FIG. 8A the access and closure system 800 is generally the same as previously disclosed in FIGS. 7A-7Z4 but with the major difference being that the piercing element and the expandable balloon are integral with the rest of the system as opposed to being separate, discrete instruments. Here, system 800 includes an elongate shaft 824 having a proximal end and a distal end. A tapered atraumatic tip 804 is coupled to the distal end of the elongate shaft 824. A retractable piercing tip 802 is coupled to the distal end of the tip 804 and is used to pierce the septal wall S.

The tapered tip 804 also includes a radially expandable member 806 disposed over the tip, here the expandable member 806 is a balloon that may be inflated to dilate the hole formed by piercing tip 802 in the septal wall S. The balloon may be in a collapsed, deflated configuration during delivery so that the device has its lowest profile. Fasteners 808 which may be any of the fasteners disclosed herein are stored in the tip 804 and have their free ends coupled to a coupling element 814 which are stored in the fastener platform 810. The platform 810 generally takes the same form as previously described above. Here the platform is in a collapsed configuration to have a low profile for delivery. Platform 810 is pivotably coupled 812 with elongate shaft 824. And similarly, as described above, pusher rod 816 may be actuated to move the platform 810 from the collapsed configuration to the expanded configuration.

Piercing elements 818 with piercing tips are stored in open channels in hub 820 and the piercing elements 818 are also slidably disposed in sheaths 822.

The elongate shaft 824, sheaths 822, piercing elements 818, piercing tip 802, pusher rob 816 all extend proximally and are coupled with a handle 826. The handle includes actuators 828, 830, 836, 838 which may be any form of actuator such as a rotatable wheel, knob, slider, lever, etc. but here they are sliders. Actuation of slider 828 advances the piercing tip 802 to penetrate the septal wall S and form an initial hole. Actuation of slider 838 controls movement of the pusher rod 816 which controls deployment of the platform 810. Actuation of slider 836 controls movement of the sheaths 822, and actuation of slider 830 controls movement of the piercing elements 818.

Handle 826 also includes a hemostasis valve 834 on the proximal end and this may be any valve such as a duck bill or Tuohy-Borst valve that prevents blood leakage or may be used to help secure instruments inserted into the proximal end of the handle. A side port 832 may be fluidly coupled with an inflation lumen in the system that can be used to inflate and deflate radially expandable member 806.

In FIG. 8A, the system 800 is advanced so the tip 804 is adjacent the right side of the septal wall S and then the piercing tip is advanced by actuating slider 828 distally thereby piercing a hole in the septal wall.

In FIG. 8B, after the hole has been pierced into the septal wall, a guidewire GW is advance through a lumen in the elongate shaft and piercing tip so the distal end of the guidewire is in the left side of the heart. The piercing tip 802 may be retracted. The platform 810 is deployed as previously described and the fasteners may be any of those disclosed previously, and they are deployed as previously described, and the other steps in FIGS. 7A-7Z4 may also be performed. FIG. 8B shows the T-tag fasteners anchored to the tissue surrounding the septal puncture and the platform has been retracted back into the collapsed configuration. Additionally, the sheaths have been retracted so they are disposed outside of the open channels in the hub and they bow out to clear a path for an interventional or diagnostic device. The tip 804 is advanced through the hole pierced in the septal wall S so that the expandable member 806 is disposed in the hole.

FIG. 8C shows expansion of expandable member 806 to dilate the hole pierced in the septal wall S by the piercing tip 802. Here, the balloon 806 is inflated via side port 832 which is fluidly coupled to the balloon. Saline, water, contrast media or other fluids may be used to inflate the balloon and dilate the hole to accommodate larger sized devices such as an interventional catheter or a diagnostic catheter to allow a left side of the heart procedure to be performed. After the hole is dilated and the procedure performed, the hole is closed or substantially closed with the fasteners in substantially the same manner previously described in FIGS. 7A-7Z4 previously described above. Therefore, FIGS. 8A-8C show an example of an access and closure system 800 that includes everything needed to create transseptal access and then close the hole after a diagnostic or interventional procedure has been performed, all in one integral system. The system is used substantially the same way as previously described above with respect to FIGS. 7A-7Z4.

Any of the procedures disclosed herein may be observed by an operator using fluoroscopy, ultrasound or any other visualization technique.

Any portion of the systems disclosed herein may remain permanently in the patient, or they may be formed from bioresorable materials so that they are resorbed into the body after healing has taken place.

### NOTES AND EXAMPLES

The following, non-limiting examples, detail certain aspects of the present subject matter to solve the challenges and provide the benefits discussed herein, among others.

Example 1 is a system for forming and closing an aperture in tissue for atrial transseptal access, the system comprising: an elongate shaft having a proximal end and a distal end; a tip coupled to the distal end of the elongate shaft; a fastener platform operably coupled to the elongate shaft and adjacent the distal end thereof, the fastener platform actuatable between a collapsed configuration and an expanded configuration, wherein in the collapsed configuration the fastener platform has a profile suitable for delivery through a patient's blood vessel to the aperture, and wherein in the expanded configuration the fastener platform extends radially outward from the elongate shaft to engage and support tissue surrounding the aperture; one or more fasteners adjacent the distal end of the elongate shaft and carried by the system, the one or more fasteners each comprising a free end and an anchor end, the anchor end configured to be anchored to the tissue surrounding the aperture, and the free end extending proximally; an anchor coupled to the anchor end of the one or more fasteners; and one or more penetrating shafts each having a tissue piercing tip, wherein the one or more penetrating shafts are disposed alongside the elongate shaft and have a retracted configuration and an extended configuration, wherein in the retracted configuration the one or more penetrating shafts have a profile suitable for delivery through the blood vessel, and wherein in the extended configuration the one or more penetrating shafts are configured to pierce the tissue surrounding the aperture and configured to either engage the free end of the one or more fasteners so the one or more fasteners may be pulled through the tissue surrounding the aperture, or configured to pierce the tissue surrounding the aperture and deliver the anchor end of the one or more fasteners through the tissue surrounding the aperture to a left side of a patient's heart for anchoring with the anchor.

Example 2 is the system of Example 1, wherein the tip is an atraumatic tip coupled to the distal end of the elongate shaft.

Example 3 is the system of any of Examples 1-2, wherein the tip is a piercing tip coupled to the distal end of the elongate shaft; wherein the piercing tip is integral with the elongate shaft and configured to form the aperture in the tissue.

Example 4 is the system of any of Examples 1-3, further comprising a piercing tip, wherein the piercing tip is an instrument discrete and separate from the elongate shaft and the tip, the piercing tip configured to form the aperture in the tissue.

Example 5 is the system of any of Examples 1-4, further comprising a radially expandable dilating member adjacent the distal end of the elongate shaft, the radially expandable dilating member having an expanded configuration and a collapsed configuration, wherein in expanded configuration the radially expandable dilating member is configured to be disposed in the aperture and expand the aperture in the tissue, and wherein in the collapsed configuration the radially expandable dilating member has a profile suitable for delivery through the patient's blood vessel to the aperture.

Example 6 is the system of any of Examples 1-5, wherein the radially expandable dilating member is adjacent the tip, and is integral with the tip or the elongate shaft.

Example 7 is the system of any of Examples 1-6, wherein the radially expandable dilating member is adjacent the tip, and wherein the radially expandable dilating member is an instrument discrete and separate from the elongate shaft and tip.

Example 8 is the system of any of Examples 1-7, wherein the radially expandable dilating member is a balloon.

Example 9 is the system of any of Examples 1-8, wherein the aperture is disposed in an atrial septum.

Example 10 is the system of any of Examples 1-9, further comprising a separate and discrete interventional device configured to be passed through the aperture to allow performance of a therapeutic or diagnostic procedure on a left side of a patient's heart.

Example 11 is the system of any of Examples 1-10, wherein the one or more fasteners are configured to be fastened to the tissue surrounding the aperture prior to introduction of the interventional device into the aperture.

Example 12 is the system of any of Examples 1-11, further comprising a lumen extending between the proximal and distal ends of the elongate shaft, the lumen sized to slidably receive a guidewire.

Example 13 is the system of any of Examples 1-12, further comprising a handle coupled to the proximal end of the elongate shaft, the handle comprising one or more actuators operably coupled with the fastener platform, the one or more fasteners, the anchor, or the one or more penetrating shafts, wherein actuation of the one or more actuators actuate the fastener platform between the expanded and collapsed configurations, or wherein actuation of the one or more actuators actuate the anchor into an expanded configuration, or wherein actuation of the one or more actuators release the one or more fasteners from the handle, or wherein actuation of the one or more actuators move the one or more penetrating shafts between the extended configuration and the retracted configuration.

Example 14 is the system of any of Examples 1-13, wherein the handle comprises an upper handle and a lower handle, the upper and lower handles releasably coupled together, and wherein the upper handle comprises at least one of the one or more actuators and the lower handle comprises at least one of the one or more actuators.

Example 15 is the system of any of Examples 1-14, wherein the one or more actuators comprises a lever, a knob, or a slide.

Example 16 is the system of any of Examples 1-15, wherein the fastener platform comprises a plurality of wings that extend radially outward from the elongate shaft when the fastener platform is in the expanded configuration.

Example 17 is the system of any of Examples 1-16, wherein the one or more fasteners comprise one or more sutures.

Example 18 is the system of any of Examples 1-17, wherein the one or more sutures comprise a looped suture having first and second free ends, and wherein the anchor is formed from the looped portion of the suture.

Example 19 is the system of any of Examples 1-18, wherein the one or more sutures comprise first and second free ends, and wherein a coupling element is attached to the first and second free ends, and wherein the coupling element is configured to be joined with the one or more penetrating shafts.

Example 20 is the system of any of Examples 1-19, wherein the one or more sutures comprise first and second free ends, and wherein a coupling element is attached to the first free end, wherein the coupling element is configured to be joined with the one or more penetrating shafts, and wherein the second free end comprises an anchor configured to prevent the second free end from pulling out of the tissue surrounding the aperture.

Example 21 is the system of any of Examples 1-20, wherein the anchor comprises a T-tag or pledget.

Example 22 is the system of any of Examples 1-21, further comprising a management sheath slidably disposed over the one or more sutures, the management sheath configured to control and protect the one or more sutures.

Example 23 is the system of any of Examples 1-22, wherein the one or more penetrating shafts comprise a needle.

Example 24 is the system of any of Examples 1-23, further comprising a securing element coupled with the one or more fasteners, the securing element configured to hold the one or more fasteners in a closed position thereby closing the aperture.

Example 25 is the system of any of Examples 1-24, wherein the securing element comprises a pre-tied knot, a clip or a crimping element.

Example 26 is the system of any of Examples 1-25, further comprising a cutting element configured to cut the securing element to a desired length.

Example 27 is the system of any of Examples 1-26, further comprising a cutting element configured to cut the one or more fasteners to a desired length.

Example 28 is a system for forming and closing an aperture in tissue for atrial transseptal access, the system comprising: an elongate shaft having a proximal end and a distal end; a piercing tip coupled to the distal end of the elongate shaft, wherein the piercing tip is configured to form the aperture in the tissue; a radially expandable dilating member adjacent the distal end of the elongate shaft and the piercing tip, the radially expandable dilating member having an expanded configuration and a collapsed configuration, wherein in expanded configuration the radially expandable dilating member is configured to expand the aperture in the tissue, and wherein in the collapsed configuration the radially expandable dilating member has a profile suitable for delivery through the patient's blood vessel to the aperture; a fastener platform operably coupled to the elongate shaft and adjacent the distal end thereof, the fastener platform actuatable between a collapsed configuration and an expanded configuration, wherein in the collapsed configuration the fastener platform has a profile suitable for delivery through a patient's blood vessel to the aperture, and wherein in the expanded configuration the fastener platform extends radially outward from the elongate shaft to engage and support tissue surrounding the aperture; one or more fasteners adjacent the distal end of the elongate shaft and carried by the system, the one or more fasteners each comprising a free end and an anchor end, the anchor end configured to be anchored to the tissue surrounding the aperture, and the free end extending proximally; an anchor coupled to the anchor end of the one or more fasteners; and one or more penetrating shafts each having a tissue piercing tip, wherein the one or more penetrating shafts are disposed alongside the elongate shaft and have a retracted configuration and an extended configuration, wherein in the retracted configuration the one or more penetrating shafts have a profile suitable for delivery through the blood vessel, and wherein in the extended configuration the one or more penetrating shafts are configured to pierce the tissue surrounding the aperture and configured to either engage the free end of the one or more fasteners so the one or more fasteners may be pulled through the tissue surrounding the aperture, or configured to pierce the tissue surrounding the aperture and deliver the anchor end of the one or more fasteners through the tissue surrounding the aperture to a left side of a patient's heart for anchoring with the anchor.

Example 29 is the system of Example 28, further comprising a separate and discrete interventional device configured to be passed through the aperture to allow performance of a therapeutic or diagnostic procedure on a left side of a patient's heart.

Example 30 is the system of any of Examples 28-29, wherein the one or more fasteners are configured to be fastened to the tissue surrounding the aperture prior to introduction of the interventional device into the aperture.

Example 31 is the system of any of Examples 28-30, further comprising a handle further comprising a handle coupled to the proximal end of the elongate shaft, the handle comprising one or more actuators operably coupled with the fastener platform, the one or more fasteners, the anchor, or the one or more penetrating shafts, wherein actuation of the one or more actuators actuate the fastener platform between the expanded and collapsed configurations, or wherein actuation of the one or more actuators actuate the anchor into an expanded configuration, or wherein actuation of the one or more actuators release the one or more fasteners from the handle, or wherein actuation of the one or more actuators move the one or more penetrating shafts between the extended configuration and the retracted configuration.

Example 32 is the system of any of Examples 28-31, wherein the one or more penetrating shafts comprise a needle.

Example 33 is the system of any of Examples 28-32, further comprising a securing element coupled with the one or more fasteners, the securing element configured to hold the one or more fasteners in a closed position thereby closing the aperture.

Example 34 is the system of any of Examples 28-33, further comprising a cutting element configured to cut the one or more fasteners to a desired length.

Example 35 is a method for forming and closing an aperture in tissue for atrial transseptal access, the method comprising: piercing an atrial septum with a piercing tip to form an aperture therethrough, the aperture extending from a right atrium to a left atrium; advancing a fastener platform in a collapsed configuration through the aperture into the left atrium; radially expanding the fastener platform from the collapsed configuration into an expanded configuration to engage and support tissue surrounding the aperture; extending one or more penetrating shafts from a retracted configuration to an extended configuration and piercing through the tissue surrounding the aperture; engaging the one or more penetrating shafts with one or more fasteners carried by the tip with the one or more penetrating shafts, or advancing one or more fasteners with the one or more penetrating shafts through the tissue into the left atrium; retracting the one or more penetrating shafts thereby pulling the one or more fasteners through the tissue surrounding the aperture into the right atrium, or retracting the one or more penetrating shafts from the left atrium; dilating the aperture by disposing a radially expandable member in the aperture and expanding the radially expandable member; passing an interventional device through the aperture and performing a transseptal interventional procedure; manipulating the one or more fasteners into a closed configuration thereby closing the aperture; and applying one or more securing elements to the one or more fasteners to hold the one or more fasteners in the closed configuration thereby maintaining closure of the aperture.

Example 36 is the method of Example 35, further comprising removing the piercing tip from the aperture to a position proximal of the aperture, before expanding the fastener platform.

Example 37 is the method of any of Examples 35-36, wherein radially expanding the fastener platform, extending the one or more penetrating shafts, or retracting the one or more penetrating shafts comprises actuating an actuator disposed on a handle operably coupled therewith.

Example 38 is the method of any of Examples 35-37, wherein the handle comprises an upper handle portion and a lower handle portion, the method further comprising separating the upper handle portion from the lower handle portion.

Example 39 is the method of any of Examples 35-38, wherein dilating the aperture comprises radially expanding a balloon in the aperture.

Example 40 is the method of any of Examples 35-39, wherein the radially expandable member is integral with the piercing tip.

Example 41 is the method of any of Examples 35-40, wherein the piercing tip is integral with the fastener platform.

Example 42 is the method of any of Examples 35-41, further comprising cutting the one or more fasteners to a desired length.

Example 43 is the method of any of Examples 35-42, further comprising removing the interventional device from the aperture before manipulating the one or more fasteners into the closed configuration and closing the aperture.

Example 44 is the method of any of Examples 35-43, wherein the one or more fasteners comprise one or more sutures, and wherein manipulating the one or more fasteners into the closed configuration comprises applying tension to the one or more sutures and fixing the one or more sutures in a tensioned configuration with a securing element.

Example 45 is the method of any of Examples 35-44, wherein the securing element comprises pre-tied knot or a clip.

Example 46 is the method of any of Examples 35-45, wherein the one or more fasteners comprise one or more sutures, the method further comprising advancing one or more management sheaths over the one or more sutures to control and protect the one or more sutures.

Example 47 is the method of any of Examples 35-46, further comprising retracting the fastener platform from the expanded configuration into the collapsed configuration.

Example 48 is the method of any of Examples 35-47, further comprising disposing the one or more fasteners radially outward and away from the aperture to provide clearance for the interventional device.

Example 49 is the method of any of Examples 35-48, further comprising observing the aperture using fluoroscopy or ultrasound imaging.

In Example 50, the apparatuses or methods of any one or any combination of Examples 1-49 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system for forming and closing an aperture in tissue for atrial transseptal access, the system comprising:
an elongate shaft having a proximal end and a distal end;
a tip coupled to the distal end of the elongate shaft;
a fastener platform operably coupled to the elongate shaft and adjacent the distal end thereof, the fastener platform actuatable between a collapsed configuration and an expanded configuration, wherein in the collapsed configuration the fastener platform has a profile suitable for delivery through a patient's blood vessel to the aperture, and wherein in the expanded configuration the fastener platform extends radially outward from the elongate shaft to engage and support tissue surrounding the aperture;
one or more fasteners adjacent the distal end of the elongate shaft and carried by the system, the one or more fasteners each comprising a free end and an anchor end, the anchor end configured to be anchored to the tissue surrounding the aperture, and the free end extending proximally;
an anchor coupled to the anchor end of the one or more fasteners; and
one or more penetrating shafts each having a tissue piercing tip, wherein the one or more penetrating shafts are disposed alongside the elongate shaft and have a retracted configuration and an extended configuration, wherein in the retracted configuration the one or more penetrating shafts have a profile suitable for delivery through the blood vessel, and wherein in the extended configuration the one or more penetrating shafts are configured to pierce the tissue surrounding the aperture and configured to either engage the free end of the one or more fasteners so the one or more fasteners may be pulled through the tissue surrounding the aperture, or configured to pierce the tissue surrounding the aperture and deliver the anchor end of the one or more fasteners through the tissue surrounding the aperture to a left side of a patient's heart for anchoring with the anchor.

2.-3. (canceled)

4. The system of claim 1, further comprising a piercing tip, wherein the piercing tip is an instrument discrete and separate from the elongate shaft and the tip, the piercing tip configured to form the aperture in the tissue.

5. The system of claim 1, further comprising a radially expandable dilating member adjacent the distal end of the elongate shaft, the radially expandable dilating member having an expanded configuration and a collapsed configuration, wherein in expanded configuration the radially expandable dilating member is configured to be disposed in the aperture and expand the aperture in the tissue, and wherein in the collapsed configuration the radially expandable dilating member has a profile suitable for delivery through the patient's blood vessel to the aperture.

6. (canceled)

7. The system of claim 5, wherein the radially expandable dilating member is adjacent the tip, and wherein the radially expandable dilating member is an instrument discrete and separate from the elongate shaft and tip.

8.-9. (canceled)

10. The system of clam 1, further comprising a separate and discrete interventional device configured to be passed through the aperture to allow performance of a therapeutic or diagnostic procedure on a left side of a patient's heart, wherein the one or more fasteners are configured to be fastened to the tissue surrounding the aperture prior to introduction of the interventional device into the aperture.

11.-12. (canceled)

13. The system of claim 1, further comprising a handle coupled to the proximal end of the elongate shaft, the handle comprising one or more actuators operably coupled with the fastener platform, the one or more fasteners, the anchor, or the one or more penetrating shafts,
wherein actuation of the one or more actuators actuate the fastener platform between the expanded and collapsed configurations, or
wherein actuation of the one or more actuators actuate the anchor into an expanded configuration, or
wherein actuation of the one or more actuators release the one or more fasteners from the handle, or
wherein actuation of the one or more actuators move the one or more penetrating shafts between the extended configuration and the retracted configuration.

14. The system of claim 13, wherein the handle comprises an upper handle and a lower handle, the upper and lower handles releasably coupled together, and wherein the upper handle comprises at least one of the one or more actuators and the lower handle comprises at least one of the one or more actuators.

15. (canceled)

16. The system of claim 1, wherein the fastener platform comprises a plurality of wings that extend radially outward from the elongate shaft when the fastener platform is in the expanded configuration.

17. The system of claim 1, wherein the one or more fasteners comprise one or more sutures.

18. The system of claim 17, wherein the one or more sutures comprise a looped suture having first and second free ends, and wherein the anchor is formed from the looped portion of the suture.

19. The system of claim 17, wherein the one or more sutures comprise first and second free ends, and wherein a coupling element is attached to the first and second free ends, and wherein the coupling element is configured to be joined with the one or more penetrating shafts.

20. The system of claim 17, wherein the one or more sutures comprise first and second free ends, and wherein a coupling element is attached to the first free end, wherein the coupling element is configured to be joined with the one or more penetrating shafts, and wherein the second free end comprises an anchor configured to prevent the second free end from pulling out of the tissue surrounding the aperture.

21. (canceled)

22. The system of claim 17, further comprising a management sheath slidably disposed over the one or more sutures, the management sheath configured to control and protect the one or more sutures.

23. The system of claim 1, wherein the one or more penetrating shafts comprise a needle.

24. The system of claim 1, further comprising a securing element coupled with the one or more fasteners, the securing element configured to hold the one or more fasteners in a closed position thereby closing the aperture.

25.-27. (canceled)

28. A system for forming and closing an aperture in tissue for atrial transseptal access, the system comprising:
an elongate shaft having a proximal end and a distal end;
a piercing tip coupled to the distal end of the elongate shaft, wherein the piercing tip is configured to form the aperture in the tissue;
a radially expandable dilating member adjacent the distal end of the elongate shaft and the piercing tip, the radially expandable dilating member having an expanded configuration and a collapsed configuration, wherein in expanded configuration the radially expandable dilating member is configured to expand the aperture in the tissue, and wherein in the collapsed configuration the radially expandable dilating member has a profile suitable for delivery through the patient's blood vessel to the aperture;
a fastener platform operably coupled to the elongate shaft and adjacent the distal end thereof, the fastener platform actuatable between a collapsed configuration and an expanded configuration, wherein in the collapsed configuration the fastener platform has a profile suitable for delivery through a patient's blood vessel to the aperture, and wherein in the expanded configuration the fastener platform extends radially outward from the elongate shaft to engage and support tissue surrounding the aperture;
one or more fasteners adjacent the distal end of the elongate shaft and carried by the system, the one or more fasteners each comprising a free end and an anchor end, the anchor end configured to be anchored to the tissue surrounding the aperture, and the free end extending proximally;
an anchor coupled to the anchor end of the one or more fasteners; and
one or more penetrating shafts each having a tissue piercing tip, wherein the one or more penetrating shafts are disposed alongside the elongate shaft and have a retracted configuration and an extended configuration, wherein in the retracted configuration the one or more penetrating shafts have a profile suitable for delivery through the blood vessel, and wherein in the extended configuration the one or more penetrating shafts are configured to pierce the tissue surrounding the aperture and configured to either engage the free end of the one or more fasteners so the one or more fasteners may be pulled through the tissue surrounding the aperture, or configured to pierce the tissue surrounding the aperture and deliver the anchor end of the one or more fasteners through the tissue surrounding the aperture to a left side of a patient's heart for anchoring with the anchor.

29.-34. (canceled)

35. A method for forming and closing an aperture in tissue for atrial transseptal access, the method comprising:
piercing an atrial septum with a piercing tip to form an aperture therethrough, the aperture extending from a right atrium to a left atrium;
advancing a fastener platform in a collapsed configuration through the aperture into the left atrium;
radially expanding the fastener platform from the collapsed configuration into an expanded configuration to engage and support tissue surrounding the aperture;
extending one or more penetrating shafts from a retracted configuration to an extended configuration and piercing through the tissue surrounding the aperture;
engaging the one or more penetrating shafts with one or more fasteners carried by the tip with the one or more penetrating shafts, or advancing one or more fasteners with the one or more penetrating shafts through the tissue into the left atrium;
retracting the one or more penetrating shafts thereby pulling the one or more fasteners through the tissue surrounding the aperture into the right atrium, or retracting the one or more penetrating shafts from the left atrium;
dilating the aperture by disposing a radially expandable member in the aperture and expanding the radially expandable member;
passing an interventional device through the aperture and performing a transseptal interventional procedure;
manipulating the one or more fasteners into a closed configuration thereby closing the aperture; and
applying one or more securing elements to the one or more fasteners to hold the one or more fasteners in the closed configuration thereby maintaining closure of the aperture.

36.-43. (canceled)

44. The method of claim 35, wherein the one or more fasteners comprise one or more sutures, and wherein manipulating the one or more fasteners into the closed configuration comprises applying tension to the one or more sutures and fixing the one or more sutures in a tensioned configuration with a securing element.

45. The method of claim 44, wherein the securing element comprises pre-tied knot or a clip.

46. The method of claim 35, wherein the one or more fasteners comprise one or more sutures, the method further comprising advancing one or more management sheaths over the one or more sutures to control and protect the one or more sutures.

47. (canceled)

48. The method of claim 35, further comprising disposing the one or more fasteners radially outward and away from the aperture to provide clearance for the interventional device.

49. (canceled)
